(19) United States
(12) Patent Application Publication
(10) Pub. No.: US 2020/0172969 A1
(43) Pub. Date: Jun. 4, 2020

(54) METHOD FOR PREDICTING DIFFERENTIATION ABILITY OF PLURIPOTENT STEM CELL, AND REAGENT FOR SAME

(71) Applicant: FOUNDATION FOR BIOMEDICAL RESEARCH AND INNOVATION AT KOBE, Kobe (JP)

(72) Inventors: Shin KAWAMATA, Kobe (JP); Takako YAMAMOTO, Kobe (JP); Chiemi TAKENAKA, Kobe (JP)

(73) Assignee: FOUNDATION FOR BIOMEDICAL RESEARCH AND INNOVATION AT KOBE, Kobe (JP)

(21) Appl. No.: 16/623,382

(22) PCT Filed: Jun. 4, 2018
(86) PCT No.: PCT/JP2018/021430

§ 371 Date: Dec. 16, 2019

(30) Foreign Application Priority Data
Jun. 19, 2017 (JP) 2017-120024
Dec. 12, 2017 (JP) 2017-237420

(51) Int. Cl. C12Q 1/6876; C12Q 1/02; C12N 5/074

(52) U.S. Cl. CPC C12Q 1/6876; C12Q 2600/158; C12N 5/0607; C12Q 1/02

(57) ABSTRACT

The invention provides a method for predicting a differentiation potential of a pluripotent stem cell comprising measuring an expression level of CHD7 of the human pluripotent stem cell. The invention also provides a method for evaluating a medium for a human pluripotent stem cell comprising measuring an expression level of CHD7 of the pluripotent stem cell.

### TECHNICAL FIELD

The present invention relates to a method for predicting differentiation potential of a pluripotent stem cell, and a reagent and a kit therefor. The present invention also relates to a method for evaluating a medium for a pluripotent stem cell, and a reagent and a kit therefor. The present invention further relates to a method for reducing or eliminating differentiation resistance of a pluripotent stem cell.

### BACKGROUND ART

Embryonic stem cell (ESC) and induced pluripotent stem cell (iPSC) are pluripotent stem cells (PSCs) having two characteristics: the ability to proliferate in an undifferentiated state (self-proliferation ability) and the potential to differentiate into three germ layer lineages (differentiation potential) in response to differentiation stimuli. However, differentiation potential cannot be verified until an external differentiation stimulus is applied. While the mechanism by which PSC stops self-proliferation and switches to the initiation of differentiation and a series of processes thereof are extremely important in understanding the biology of PSC, they have not been fully elucidated.

This problem is particularly important when cell therapy using PSC-derived cells is to be performed. The inclusion of undifferentiated cells in the final product of PSC-derived differentiated cells creates a risk of tumorigenesis. Therefore, it is clinically highly valuable to understand the process of the initiation of differentiation.

The group of Okano and Yamanaka et al. disclosed a method for selecting iPSC-derived differentiated cells with a reduced risk of tumorigenesis by inducing secondary neurosphere (SNS) from iPSCs and using Nanog gene expression in the SNS as an index (patent document 1). Furthermore, the group considered that the differentiation resistance of iPSCs is an inherent property of the iPSC clone rather than the property due to differentiation-inducing conditions, and disclosed a method for evaluating differentiation resistance of iPSC by using the expression of Nanog gene in SNS derived from the iPSCs as an index (patent document 2).

However, all of these methods require time and labor to once differentiate iPSCs into SNS for evaluation of the differentiation resistance. If a marker capable of predicting differentiation resistance while PSCs are in an undifferentiated state is found, a safe cell therapy agent without a risk of tumorigenesis can be provided quickly and easily. However, such differentiation marker has not yet been reported.

### DOCUMENT LIST

### Patent Documents

patent document 1: Japanese Translation of PCT International Application Publication No. 2011-530273

patent document 2: Japanese Translation of PCT International Application Publication No. 2012-527887

### SUMMARY OF THE INVENTION

### Problems to be Solved by the Invention

An object of the present invention is to provide a marker capable of predicting differentiation resistance of a pluripotent stem cell (PSC) while it is in an undifferentiated state, and provide a method for rapidly evaluating differentiation potential of the pluripotent stem cell by using expression of the marker as an index.

Another object of the present invention is to provide a means for searching for culture conditions suitable for maintaining the differentiation potential of a pluripotent stem cell by using expression of the aforementioned differentiation resistance prediction marker as an index.

A still another object of the present invention is to provide a method for evaluating a medium for a pluripotent stem cell by using expression of the aforementioned differentiation resistance prediction marker as an index.

A further object of the present invention is to provide a method for reducing or eliminating differentiation resistance of a pluripotent stem cell.

### Means of Solving the Problems

PSC shows resistance to differentiation if it acquires genetic abnormality during long-term culture or the reprogramming process in the case of iPSC. Genetic abnormality can be tested in a timely manner by using the state-of-the-art sequencing techniques, and PSC and PSC derivative used in cell therapy can be frequently subjected to genetic screening to eliminate genetically abnormal cells. However, even PSC with a normal karyotype sometimes shows differentiation resistance caused by epigenetic modification due to culture conditions, and it is necessary to regularly confirm the quality of PSC by testing the differentiation potential of the PSC by embryoid body (EB) formation assay or cytokine-induced differentiation assay.

In the course of performing an EB formation assay of PSCs, the present inventors have found that certain culture conditions confer differentiation resistance to both ESCs and iPSCs. That is, when PSCs were cultured with commercially available Essential 8 medium (Thermo Fisher Scientific, hereinafter to be also referred to as “Es8”) or Stem-Partner (registered trade mark) Human iPS/ES cells medium (KYOKUTO PHARMACEUTICAL INDUSTRIAL CO., LTD., hereinafter to be also referred to as “SPM”) (Takenaka et al., PLoS One 10(6), 2015), EBs were formed. However, when the PSCs were transferred to a commercially available ReproFF2 medium (REPROCELL, hereinafter to be also referred to as “RFF2”) and cultured for 5 passages or more, it lost differentiation potential and no longer formed EBs. When the PSCs were placed back in Es8 or SPM and further cultured for 5 passages or more, the differentiation potential was recovered. The present inventors have found Chromodomain Helicase DNA binding protein 7 (hereinafter to be also referred to as “CHD7”) as one of the gene candidates relating to the reversible alteration in the differentiation potential of PSC based on the analyses by principal component analysis (hereinafter to be also referred to as PCA) and GeneChip analysis. The effect of PSC culture with Es8 and RFF2 on the expression of CHD7 gene was examined to find that the expression of CHD7 gene was remarkably suppressed by culturing with RFF2, whereas CHD7 gene showed a moderate expression level in PSCs by culturing with Es8. When CHD7 mRNA was introduced into PSCs cultured with RFF2 and CHD7 was upregulated, PSCs spontaneously initiated to differentiate, and undifferentiated cell maintaining culture system did not support proliferation of differentiated cells. On the other hand, when CHD7 siRNA was introduced into PSCs cultured with Es8 and CHD7 was down-regulated, the differentiation potential of PSCs were partially impaired. That is, it was clarified that PSC can proliferate in an undifferentiated state if CHD7 expression does not exceed a certain upper limit; however, when the expression falls below a threshold value level, PSC no longer responds to differentiation stimuli, and PSC having a CHD7 expression range between the upper limit and the threshold value maintains the property that can respond to differentiation stimuli. From the above results, the present inventors have confirmed that differentiation potential/differentiation resistance of PSC can be predicted using the expression level of CHD7 in PSC in an undifferentiated state as an index, which resulted in the completion of the present invention.

Accordingly, the present invention provides the following.

[1] A method for predicting a differentiation potential of a pluripotent stem cell comprising measuring an expression level of CHD7 of the human pluripotent stem cell.
[2] The method of [1], wherein a human pluripotent stem cell having aforementioned expression level of CHD7 of not less than 1500 copies in 5 ng of the total RNA is predicted to show a differentiation potential in response to a differentiation stimulus.
[3] The method of [2], wherein the aforementioned expression level of CHD7 is not less than 2710 copies in 5 ng of the total RNA.
[4] The method of [2] or [3], wherein the aforementioned expression level of CHD7 is an expression level of a human pluripotent stem cell cultured for not less than 5 passages with Essential 8 medium or Stem-Partner (registered trade mark) Human iPS/ES cells medium.
[5] The method of any of [1] to [4], wherein the aforementioned human pluripotent stem cell is an embryonic stem cell or an induced pluripotent stem cell.
[6] A method for evaluating a medium for a human pluripotent stem cell comprising measuring an expression level of CHD7 of the pluripotent stem cell.
[7] The method of [6], wherein the aforementioned human pluripotent stem cell is a human pluripotent stem cell cultured for not less than 5 passages with the test medium.
[8] The method of [6] or [7], wherein the test medium is evaluated as being capable of maintaining the human pluripotent stem cell to show a differentiation potential in response to a differentiation stimulus, when the aforementioned expression level of CHD7 is not less than 1500 copies in 5 ng of the total RNA.
[9] The method of [8], wherein the aforementioned expression level of CHD7 is not less than 2710 copies in 5 ng of the total RNA.
[10] The method of any of [6] to [9], wherein the aforementioned human pluripotent stem cell is an embryonic stem cell or an induced pluripotent stem cell.
[11] A reagent or kit for predicting a differentiation potential of a human pluripotent stem cell and/or evaluating a medium for human pluripotent stem cell, comprising a substance capable of detecting an expression of CHD7.
[12] A differentiation-inducing agent for a human pluripotent stem cell comprising a nucleic acid encoding CHD7.
[13] The method of [1], wherein the human pluripotent stem cell is predicted to show a differentiation potential in response to a differentiation stimulus when the aforementioned expression level of CHD7 is a protein level not less than two times a CHD7 protein level of a human pluripotent stem cell showing a differentiation resistance.
[14] The method of [13], wherein the aforementioned expression level of CHD7 is an expression level of a human pluripotent stem cell cultured for not less than 5 passages with Essential 8 medium or Stem-Partner (registered trade mark) Human iPS/ES cells medium.
[15] The method of [13] or [14] wherein the aforementioned human pluripotent stem cell showing the differentiation resistance is a human pluripotent stem cell cultured for not less than 5 passages with ReproFF2 medium.

### Effect of the Invention

According to the present invention, whether a human pluripotent stem cell (PSC) shows a differentiation potential in response to a differentiation stimulus can be predicted in an undifferentiated state before applying a differentiation stimulus. In addition, culture conditions suitable for maintaining a human pluripotent stem cell in a state holding a property showing a differentiation potential in response to a differentiation stimulus can be found. Furthermore, a medium suitable for culturing a human pluripotent stem cell can be evaluated by measuring an expression level of CHD7 of a human pluripotent stem cell. Moreover, differentiation resistant can be reduced or eliminated by increasing an expression level of CHD7 (selecting cell with high expression) in a human pluripotent stem cell, particularly iPSC population.

### BRIEF DESCRIPTION OF THE DRAWINGS

FIG. 1 shows the differentiation potential of PSC altered depending on the culture conditions. KhES-1 cells in a single cell suspension were seeded on a dish coated with rhVitronectin-N (Thermo Fisher Scientific, hereinafter to be also referred to as “VNT-N”), and cultured for 5 passages with Essential 8 medium (hereinafter also referred to as “Es8”) (upper left photograph). The cells were then collected for embryoid body (EB) formation (lower left photograph) or transferred to ReproFF2 medium (RFF2) (upper center photograph). KhES-1 cells were cultured for 5 passages and collected for EB formation assay (lower center photograph) or transferred again to Es8 (upper right photograph). KhES-1 cells were cultured for 5 passages and EB formation assay was performed (lower right photograph). Photographs of the KhES-1 culture using either Es8 or RFF2 medium on the first day of culture (upper) and photographs of EB on the 14th day (lower) are shown. The gene expression profiles of the cells under the aforementioned culture conditions were determined by qRT-PCR scorecard panel and shown under the photographs (scale bar: 1.0 mm).

FIG. 2 shows the potential of iPSCs to EB formation altered depending on the culture conditions. iPSCs (PFX #9) in a single cell suspension were seeded on a dish coated with VNT-N and cultured for 5 passages with Es8 (upper left photograph). The cells were then collected for EB formation assay (lower left photograph) or transferred to ReproFF2 medium (RFF2) (upper center photograph). PFX #9 cells were cultured for 5 passages and collected for EB formation assay (lower center photograph) or transferred again to Es8 (upper right photograph). PFX #9 cells were cultured for 5 passages and EB formation assay was performed (lower right photograph). Photographs of the PFX #9 culture using either Es8 or RFF2 medium on the first day of culture (upper) and photographs of EB on the 14th day in EB formation assay (lower) are shown. The gene expression profiles of the cells under the aforementioned culture conditions were determined by qRT-PCR scorecard panel and shown under the photographs (scale bar: 1.0 mm).

FIG. 3 shows comparison of methylation of PSCs cultured under various conditions. A. The methylation status of the cells were determined by Illumina Human Methylation Bead Chip. Average methylation scores of 6 PSC samples (3 samples of iPSC (PFX #9) and 3 samples of ESC (KhES-1, H9)) cultured with RFF2 are shown. The score was determined by comparison with an average of 6 PSC samples (1 sample of PFX #9 cultured with SPM, 1 sample of KhES-1 cultured with SPM, 1 sample of H9 cultured with SPM, 1 sample of PFX #9 cultured with Es8, 2 samples of H9 cultured with Es8) with SPM or Es8. The number of genes in the promoter region or genes in all regions that showed the methylation score exceeding 0.2 by culturing with RFF2 or SPM and Es8 is shown in Venn diagram. B. shows clustering of methylation patterns in the promoter region of PSC cultured with RFF2, SPM or Es8. lane #1-3: the results of iPSCs (PFX #9) cultured with RFF2 medium, lane #4-6: the results of ESCs (KhES-1) cultured with RFF2 from 6 independent experiments, lane #7: the results of PFX #9 cells with SPM, lane #8: the results of KhES-1 cells with SPM, lane #9: the results of H9 ESCs with SPM, lane #10: the results of PFX #9 cell with Es8, lane #11, 12: the results of H9 with Es8 from 6 independent experiments.

FIG. 4 shows gene expression of CHD7 and self-growth factors POU5F1, SOX2, NANOG and EP300 in KhES-1 cultured with Es8 (P5 and P15) or RFF2 medium (P9 and P18) as determined by qRT-PCR. The relative amount is shown in bar graphs with the expression level in P5 using Es8 as 1. The passage numbers of (P) were added to the bars.

FIG. 5 shows schematic diagrams of CHD7 isoform 1, isoform 2 and isoform X4 and mRNA transcripts. A. CHD7 isoform 1, isoform 2 and isoform X4 having function domains and the location of PCR primer set are shown. B. CHD7 mRNA transcript (isoform 2), dominant negative transcripts and the target domains of siRNA used for evaluation of the function of CHD7 are shown.

FIG. 6 shows CHD7 isoforms detected by Western blotting. Whole cell lysates (5.3 μg) from KhES-1 cells cultured with Es8 (P11) or RFF2 (P21) were applied to each lane. Antibodies against human CHD7 were used to detect CHD7 isoform 1 (expected mass 336 kDa), isoform 2 (110 kDa) and isoform X4 (183 kDa). Signal was visualized with horseradish peroxidase.

FIG. 7 shows down regulation of CHD7 by siCHD7 transfection. A. Illustration of the protocol for EB formation and siCHD7 transfection. 2×10⁵ KhES-1 cells were seeded on a 6-well dish coated with rhVitronectin-N (recombinant human vitronectin-N) (hereinafter to be also referred to as “VTN-N”) and cultured with Es8 on day −1. Cells were transfected with small double-stranded interfering RNA against CHD7 (siCHD7) or non-specific control siRNA (mock) (day 0). After 24 hr from the transfection of siRNA, the cells were transferred to a low-attachment plate, cultured for 24 hr with Essential 6 (Es6) supplemented with ROCK (Rho-associated protein kinase) inhibitor (RI). The medium was changed to fresh Es6 medium and the cells were further cultured for 72 hr. B. Dependence of the introduced amount of siCHD7 on CHD7 gene expression level is shown. C. CHD7 gene expression level after siRNA introduction is shown. Expression of CHD7 or CHD7 in KhES-1 cells transfected with siCHD7 or control siRNA (mock) or non-transfected cells was determined by qRT-PCR (time course sampling, left) or Western blotting (sampled on day 2, right). The gene expression of CHD7 was normalized by the average of CHD7 expression measured independently three times in KhES-1 cells cultured with Es8. The representative datasets obtained from 3 independent experiments are shown.

FIG. 8 shows photographs of non-transfected EBs (upper panel), siCHD7-transfected KhES-1 cells (middle panel), and control siRNA-transfected KhES-1 cells (mock, lower panel) on day 4, day 5 and day 14. The gene expression profiles of KhES-1 cells under the specified conditions determined by qRT-PCR scorecard panel are shown under the corresponding photographs (scale bar: 1.0 mm). The representative datasets obtained from 3 independent experiments are shown.

FIG. 9 shows that downregulation of CHD7 supports the survival of ESCs cultured with nutrient-depleted medium. A. Illustration of a protocol for differentiation induction by nutrient-depleted Es8 and transfection of siCHD7. On day 0, cells were transfected with siCHD7 or control siRNA (mock) after medium (Es8) change. The medium was changed every 2 days (days 2 and 4). For cell counting and determination of gene expression by qRT-PCR, the cells were harvested 42 hr (day 2), 72 hr and 96 hr after transfection of siRNA. In the case of normal culture, ESCs were cultured with Es8. The medium was changed every day, and passage was performed every 3 days to maintain an undifferentiated state. B. Gene expression of CHD7 in KhES-1 cells transfected with siCHD7 or control siRNA (mock) or non-untransfected cells on day 0, day 1, day 2, day 3 and day 4 is shown. The gene expression of CHD7 was normalized by the average of CHD7 expression measured by qRT-PCR independently three times in KhES-1 cells cultured with Es8. The representative datasets obtained from 3 independent experiments are shown. C. Line graph plotting the number of non-transfected KhES-1 cells (non-transfected), siCHD7-transfected KhES-1 cells (siCHD7), and mock-transfected KhES-1 cells (mock) is shown. The representative datasets obtained from 3 independent experiments are shown.

FIG. 10 shows photographs of non-transfected regularly cultured KhES-1 cells (upper panels, medium changed every day), siCHD7-transfected KhES-1 cells (middle panels, medium changed every 2 days) and control siRNA-transfected KhES-1 cells (mock) (lower panels, medium changed every 2 days), each on day 2, day 3 and day 4. The gene expression profiles determined by qRT-PCR scorecard panel of KhES-1 are shown under each photograph (scale bar: 1 mm).

FIG. 11 shows that upregulation of CHD7 isoform 2 induces “spontaneous” differentiation in ESCs cultured with RFF2. A. Protocol for cell culture and transfection with mCHD7 is shown. On day 0 and day 1, KhES-1 cells were transfected with mCHD7 or control mRNA (mock) (2 times in total). On day 2, the cells were passaged, reseeded on a 6-well plate at 2×10⁵ cells/well, and further cultured for 24 hr. B. The gene expression of CHD7 after introduction of mCHD7 or control mRNA (mock) (day 0) and CHD7 were determined by qRT-PCR (time course sampling, left) and Western blotting (sampled on day 3, right). The gene expression of CHD7 was normalized by the average of CHD7 expression measured independently three times in KhES-1 cells cultured with RFF2 medium (NT: non-transfected control). C. A graph showing the proliferation of transfected or non-transfected KhES-1 cells is shown. The proliferation of mock-transfected KhES-1 cells (mock) is comparable to that of non-transfected control (non-transfected). On the other hand, proliferation of mCHD7-transfected KhES-1 cells (mCHD7) decreased on day 2 and day 3. The representative datasets obtained from 3 independent experiments are shown.

FIG. 12 shows photographs of non-transfected KhES-1 cells (upper panels), mCHD7-transfected KhES-1 cells (middle panels) and control mRNA-transfected KhES-1 cells (lower panels, mock), each on day 1, day 2 and day 3 cultured with RFF2 medium. Their gene expression profiles determined by qRT-PCR scorecard panel are shown under the photographs. The representative datasets obtained from 3 independent experiments are shown (scale bar: 1 mm).

FIG. 13 shows that transfection of CHD7 dominant negative (DN) mRNA transcript inhibits or reduces differentiation potential and cell proliferation in ESCs. A. Illustration of the protocol for transfection of CHD7 DN mRNA transcript and EB formation assay is shown. CHD7 DN1 is a transcript of chromodomain mRNA and CHD7 DN2 is a transcript of SANT-SLIDE domain mRNA (FIG. 5B). On day 0, transcripts were transfected into KhES-1 cells, the cells were transferred to low-attachment plates 24 hr later and subsequently cultured for 24 hr for EB formation with Es6 medium containing ROCK Inhibitor (RI). Microscopic observation of EBs and analysis of gene expression profile by qRT-PCR scorecard panel were performed on day 3 after DN mRNA transfection. B. Graphs showing CHD7 DN1 and CHD7 DN2 expression levels determined by qRT-PCR. C. Photographs of EBs derived from non-transfected KhES-1 cells, CHD7 DN1-transfected KhES-1 cells, CHD7 DN2-transfected KhES-1 cells, CHD7 DN1- and CHD7 DN2-transfected KhES-1 cells, and mock mRNA-transfected KhES-1 cells on day 3.

FIG. 14 shows that ESC overexpressing CHD7 could not be generated in culture using Es8. A. protocol for cell culture and transfection of CHD7 isoform 2 is shown. B. CHD7 expression on day 1 and day 2 was evaluated by qRT-PCR. The gene expression of CHD7 was normalized by the average of CHD7 expression measured independently three times in KhES-1 cells cultured with Es8. C. Photographs showing the number and state of non-transfected KhES-1 cells and CHD7- or mock-transfected KhES-1 cells on day 1 and day 2. The number of cells in one well is shown in the upper right corner of the photographs.

FIG. 15 shows that downregulation of CHD7 disrupted proliferation of ESCs cultured with Es8. A. The protocol for siCHD7 transfection is shown. KhES-1 cells were transfected with siCHD7 or nonspecific control siRNA (mock) on day 0 and day 1. The medium was changed every day (MC). On days 0-3, the cells were harvested for cell counting and CHD7 expression was determined by qRT-PCR. B. The CHD7 gene expression in siCHD7- or control siRNA (mock)-transfected KhES-1 cells, and non-transfected KhES-1 cells was determined by qRT-PCR. The gene expression of CHD7 was normalized by the average of CHD7 expression measured independently three times in KhES-1 cells cultured with Es8. C. Photographs of non-transfected KhES-1 cells (upper panel), and siCHD7-transfected KhES-1 cells (middle panel) or control siRNA-transfected KhES-1 cells (mock, lower panel) KhES-1 cells on day 3 are shown. The number of cells is shown in the upper right corner of the photographs. D. Graph showing the cell numbers.

FIG. 16 shows the gene expression profile of human CHD7 based on GeneChip data, and shows that the CHD7 level mediated differentiation potential of PSCs. Average human CHD7 expression during early embryogenic development of three different samples (Embryos before implantation) was compared with expression of human CHD7 of human PSCs cultured under various conditions (Human PSCs on feeder or feeder-less) and expression of human CHD7 of EBs on day 14 (EB at day 14). All samples were normalized by MAS5 method.

FIG. 17 shows copy number of CHD7 of PSC before EB formation and the gene expression profile of EBs on day 14. ESC (H9, KhES-1) or iPSC (PFX #9, 201B7, SHh #2) was cultured on feeder cells and with hPSC medium, or seeded and cultured in single cells on VTN-N and with Es8 (hereinafter to be also referred to as “Single-cell suspension method”). The copy number of CHD7 isoform 1 mRNA before EB formation was detected by digital PCR. The gene expression profile of EB derived from each cell on day 14 was determined by qRT-PCR scorecard panel and shown in a bar graph (N: VTN-N-coated dish).

FIG. 18 shows copy number of CHD7 mRNA of iPSCs with lower copy number of CHD7 mRNA and the gene expression profile of EB on day 14. 201B7 or PFX #9 cells were cultured feeder cell-free in single cells on VTN-N-coated dish. The copy number of CHD7 mRNA was reduced by deliberately creating an overgrowth state. The medium used was Es8. The copy number of CHD7 mRNA before EB formation was detected by digital PCR. The gene expression profile of EB derived from each cell on day 14 was determined by qRT-PCR scorecard panel and shown in a bar graph (P: passage numbers).

FIG. 19 shows the expression level of CDH7 protein in PSCs cultured under various culture conditions. P1 is H9 cultured for 17 passages with Es8, P2 is KhES-1 cultured for 10 passages with Es8, and N is KhES-1 cultured for 11 passages with RFF2. N2 (negative control) is a concentrated lysate of H9 cells cultured for 17 passages with RFF2. All cells were cultured feeder cell-free in single cells on a VTN-N-coated dish, then lysate was prepared, with the standard (concentrated lysate of H9 cells cultured for 17 passages with Es8) as 1000 units/mL, the concentrations of the primary antibody and the secondary antibody were changed and the relative values were shown in a bar graph.

FIG. 20 is an asymptote determined from the protein concentration and the number of mRNA copies.

### DESCRIPTION OF EMBODIMENTS

The present invention provides a method for predicting a differentiation potential of a pluripotent stem cell (hPSC) including measuring an expression level of CHD7 of the human pluripotent stem cell (hereinafter to be also referred to as “the prediction method of the present invention”).

In the present specification, the “differentiation potential” of the pluripotent stem cell means a potential of a pluripotent stem cell to differentiate into a three germ layer lineage or particular cell line spontaneously or in response to a particular differentiation stimulus. In a preferable embodiment of the present invention, a human pluripotent stem cell having a potential to differentiate into a cell line corresponding to a certain particular differentiation stimulus in response to the differentiation stimulus is predicted and selected. Such differentiation stimulus is not particularly limited as long as it is a stimulus that causes escape of PSC from an undifferentiated state and induction thereof into any differentiated cell, and the direction of the differentiation is known. For example, the culture conditions used for EB formation assay or cytokine-induced differentiation assay in the below-mentioned Examples and the like can be mentioned.

In the present specification, unless particularly indicated, the “expression level of CHD7” may mean any of the expression level of CHD7 gene and the expression level of CHD protein.

The present invention is based on, at least partially, the finding that the level of CHD7 expression required to maintain hPSC in an undifferentiated state has an upper limit but no lower limit, and the upper limit varies depending on the culture conditions. When the expression level of CHD7 of hPSC exceeds the upper limit, hPSC spontaneously initiates differentiation even in a maintenance medium of PSC and the culture system can no longer support proliferation of the differentiated cells. On the other hand, when the expression level of CHD7 of hPSC is lower than the threshold value, hPSC cannot show a sufficient differentiation potential in response to a differentiation stimulus and is maintained in an undifferentiated state. For hPSC to show a differentiation potential in response to a differentiation stimulus, therefore, it is necessary that the expression level of CHD7 is moderate in the PSC (i.e., not more than the above-mentioned upper limit and not less than the above-mentioned threshold value).

As mentioned above, the upper limit of the CHD7 expression level necessary for maintaining hPSC in an undifferentiated state is considered to vary depending on the culture conditions. When the expression level of CHD7 exceeds the upper limit under respective culture conditions, hPSC spontaneously initiates differentiation and maintenance and proliferation of the PSC becomes impossible under such culture conditions. If hPSC does not differentiate spontaneously, it means that the expression level has not exceeded the upper limit under the culture conditions. Therefore, it is not necessary to set the upper limit of the CHD7 expression level necessary for maintaining hPSC in an undifferentiated state under various culture conditions.

On the other hand, the threshold value of CHD7 expression level necessary for hPSC to show a differentiation potential in response to a differentiation stimulus when, for example, measuring the expression level of CHD7 gene by quantitative digital PCR analysis is, for example, not less than 2710 copies in 5 ng of the total RNA. When cultured in single cells, feeder cell-free, on a dish coated with extracellular substrate, it is unlimitatively, for example, not less than 1502 copies or not less than 1500 copies in 5 ng of the total RNA. When hPSC is ESC and cultured with feeder cells by the Small Cell Clumps method, it is unlimitatively, for example, not less than 2710 copies or not less than 2120 copies; when cultured in single cells, feeder cell-free, on a dish coated with extracellular substrate, it is unlimitatively, for example, not less than 3280 copies; when hPSC is iPSC and cultured with feeder cells by the Small Cell Clumps method, it is unlimitatively, for example, not less than 3080 copies or not less than 2280 copies; and when cultured in single cells, feeder cell-free, on a dish coated with extracellular substrate, it is unlimitatively, for example, not less than 1502 copies or not less than 1500 copies.

When the expression level of CHD7 protein is measured, the threshold value is, for example, not less than 2.0 times, not less than 3.0 times, not less than 4.0 times, not less than 5.0 times, not less than 6.0 times, not less than 7.0 times, not less than 7.7 times, not less than 8.0 times, not less than 8.5 times, not less than 9.0 times, not less than 9.2 times, not less than 10 times, not less than 10.2 times or not less than 10.3 times, compared to CHD7 protein concentration of human pluripotent stem cell showing differentiation resistance. Examples of the human pluripotent stem cell showing differentiation resistance include human pluripotent stem cell cultured for not less than 5 passages with ReproFF2 medium. Furthermore, when similar to the above-mentioned measurement, the threshold value is, for example, not more than 90.3%, not more than 90.2%, not more than 90%, not more than 89.1%, not more than 88.3%, not more than 87.0%, not more than 85.8%, not more than 85%, not more than 83.4%, not more than 80.2%, not more than 80%, not more than 75%, not more than 70%, not more than 50%, compared to CHD7 protein concentration of human pluripotent stem cell showing normal differentiation potential. Examples of the human pluripotent stem cell showing normal differentiation potential include human pluripotent stem cell cultured for not less than 5 passages with Es8 or SPM medium.

For example, the threshold value may be determined in consideration of the expression level of CHD7 gene measured by quantitative digital PCR analysis. Examples of the threshold value in consideration of the expression level of CHD7 gene include not less than 2 times, not less than 3 times, not less than 4 times, not less than 5 times and not less than 10 times, compared to CHD7 protein concentration of human pluripotent stem cell showing differentiation resistance. Examples of the human pluripotent stem cell showing differentiation resistance include human pluripotent stem cell cultured for not less than 5 passages with ReproFF2 medium. Furthermore, when similar to the above-mentioned case, the threshold value is, for example, not more than 50%, not more than 70%, not more than 75%, not more than 80% or not more than 90%, compared to CHD7 protein concentration of human pluripotent stem cell showing normal differentiation potential. Examples of the human pluripotent stem cell showing normal differentiation potential include human pluripotent stem cell cultured for not less than 5 passages with Es8 or SPM medium.

The CHD7 protein concentration may be a value obtained by directly measuring the CHD7 protein concentration of the lysate of the cultured cells, or may be a value relative to the lysate of the cultured cells as the standard. The cultured cell may be a stem cell showing differentiation resistance or a stem cell showing normal differentiation potential, and the lysate may or may not be concentrated.

In the prediction method of the present invention, a human pluripotent stem cell whose differentiation potential is predictable is not particularly limited as long as it is an undifferentiated cell having “self-proliferation ability” that enables proliferation while maintaining an undifferentiated state, and “differentiation potential” that enables differentiation into all three germ layer lineages. Examples include embryonic stem cell (ES cell), induced pluripotent stem cell (iPS cell), embryonic germ (EG) cell derived from a primordial germ cell, multipotent germline stem (mGS) cell isolated in the process of establishment and culture of GS cell from testis tissue and the like. The ES cell may be ES cell produced from a somatic cell by nuclear reprogramming (nt ES cell). Preferred is ES cell or iPS cell. The prediction method of the present invention is particularly preferably used in human pluripotent stem cell, and is applicable to any mammal in which pluripotent stem cell has been established or can be established. As the non-human mammal, for example, mouse, monkey, swine, rat, dog and the like can be mentioned.

Pluripotent stem cell can be produced by a method known per se. For example, for iPS cell, the methods described in WO2007/069666, WO2008/118820, WO2009/007852, WO2009/032194, WO2009/058413, WO2009/057831, WO2009/075119, WO2009/079007, WO2009/091659, WO2009/101084, WO2009/101407, WO2009/102983, WO2009/114949, WO2009/117439, WO2009/126250, WO2009/126251, WO2009/126655, WO2009/157593, WO2010/009015, WO2010/033906, WO2010/033920, WO2010/042800, WO2010/050626, WO 2010/056831, WO2010/068955, WO2010/098419, WO2010/102267, WO 2010/111409, WO 2010/111422, WO2010/115050, WO2010/124290, WO2010/147395, WO2010/147612, Huangfu D, et al. (2008), Nat. Biotechnol., 26: 795-797, Shi Y, et al. (2008), Cell Stem Cell, 2: 525-528, Eminli S, et al. (2008), Stem Cells. 26:2467-2474, Huangfu D, et al. (2008), Nat Biotechnol. 26:1269-1275, Shi Y, et al. (2008), Cell Stem Cell, 3, 568-574, Zhao Y, et al. (2008), Cell Stem Cell, 3:475-479, Marson A, (2008), Cell Stem Cell, 3, 132-135, Feng B, et al. (2009), Nat Cell Biol. 11:197-203, R. L. Judson et al., (2009), Nat. Biotech., 27:459-461, Lyssiotis C A, et al. (2009), Proc Natl Acad Sci USA. 106:8912-8917, Kim J B, et al. (2009), Nature. 461:649-643, Ichida J K, et al. (2009), Cell Stem Cell. 5:491-503, Heng J C, et al. (2010), Cell Stem Cell. 6:167-74, Han J, et al. (2010), Nature. 463:1096-100, Mali P, et al. (2010), Stem Cells. 28:713-720, Maekawa M, et al. (2011), Nature. 474:225-9 and the like can be mentioned.

ES cell can be established by removing an inner cell mass from the blastocyst of a fertilized egg of a target animal, and culturing the inner cell mass on fibroblast feeder cells. In addition, the cell can be maintained by passage culture with a culture medium added with substances such as leukemia inhibitory factor (LIF), basic fibroblast growth factor (bFGF) and the like. The methods of establishment and maintenance of human and monkey ES cells are described in, for example, U.S. Pat. No. 5,843,780; Thomson J A, et al. (1995), Proc Natl. Acad. Sci. USA. 92:7844-7848; Thomson J A, et al. (1998), Science. 282:1145-1147; H. Suemori et al. (2006), Biochem. Biophys. Res. Commun., 345:926-932; M. Ueno et al. (2006), Proc. Natl. Acad. Sci. USA, 103:9554-9559; H. Suemori et al. (2001), Dev. Dyn., 222:273-279; H. Kawasaki et al. (2002), Proc. Natl. Acad. Sci. USA, 99:1580-1585; Klimanskaya I, et al. (2006), Nature. 444:481-485 and the like. As for human ES cell line, for example, WA01(H1) and WA09(H9) are available from WiCell Research Institute, and KhES-1, KhES-2 and KhES-3 are available from Institute for Frontier Medical Sciences, Kyoto University (Kyoto, Japan).

EG cell can be established by culturing a primordial germ cell in the presence of a substance such as LIF, bFGF, a stem cell factor and the like (Y. Matsui et al. (1992), Cell, 70:841-847; J. L. Resnick et al. (1992), Nature, 359:550-551).

For production of an nt ES cell, a combination of the nuclear transplantation technique (J. B. Cibelli et al. (1998), Nature Biotechnol., 16:642-646) and the ES cell production technique (mentioned above) is used (Kiyoka Wakayama et al., (2008), Experimental Medicine, Vol. 26, No. 5 (Suppl.), pp. 47-52). In nuclear transplantation, reprogramming can be performed by injecting the nucleus of a somatic cell to an enucleated unfertilized egg of a mammal, and culturing for a few hours.

The mGS cell can be produced from a testis cell according to the method described in WO 2005/100548.

PSC produced as described above, preferably hPSC, may preferably contain a step of maintaining and culturing PSC prior to the measurement of the expression level of CHD7. The culture may be suspension culture or adhesive culture using a coated culture dish. In this step, adhesive culture is preferably used. PSC can be dissociated, for example, physically, or dissociated using a dissociation solution having protease activity and collagenase activity (e.g., Accutase™, Accumax™ and the like) or a dissociation solution having collagenase activity alone, or Trypsin/EDTA. Preferably, a method of dissociating cells by using Trypsin/EDTA is used.

When detaching PSC, it is preferable to contain a Rho-associated protein kinase (ROCK) inhibitor in the medium. The ROCK inhibitor is not particularly limited as long as it can suppress the function of ROCK. For example, Y-27632 can be preferably used in the present invention. The concentration of Y-27632 in the medium is not particularly limited and is preferably 1 μM-50 μM, for example, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM or 50 μM, but it is not limited to these.

The period during which the ROCK inhibitor is added to the medium may be the culture period in the step of culturing PSC, or any period that suppresses cell death at the time of single dispersion, for example, at least one day.

In the present specification, the suspension culture is forming an embryoid body by culturing cells in a non-adhesive state on a culture dish, and is not particularly limited. It can be performed using a culture dish free from an artificial treatment to improve adhesiveness to cells (e.g., coating treatment with an extracellular substrate or the like) or a culture dish that has been artificially treated to inhibit adhesion (e.g., coating treatment with polyhydroxyethylmethacrylic acid (poly-HEMA)).

In the present specification, adhesive culture can be performed by culturing on feeder cells or using a culture vessel coating treated with an extracellular substrate. The coating treatment can be performed by placing a solution containing an extracellular substrate in a culture vessel and thereafter removing the solution as appropriate.

In the present specification, the feeder cell means other cell that plays an auxiliary role and is used for adjusting the culture conditions of the target cell. In the present invention, the extracellular substrate is a supramolecular structure existing outside the cell and may be naturally derived or an artifact (recombinant). For example, substances such as vitronectin, collagen, proteoglycan, fibronectin, hyaluronic acid, tenascin, entactin, elastin, fibrillin, laminin and fragments thereof can be mentioned, and vitronectin or a fragment thereof is preferable.

PSC produced as described above, preferably hPSC, can be maintained, for example, by adhesive culture using a culture vessel coated with an extracellular substrate. The culture medium used in the step of culturing PSC can be prepared using a medium used for culturing animal cells as a basal medium. Examples of the basal medium include IMDM medium, Medium 199, Eagle's Minimum Essential Medium (EMEM), αMEM medium, Dulbecco's modified Eagle's Medium (DMEM), Ham's F12 medium, RPMI 1640 medium, Fischer's medium and a mixed medium of these and the like. As a commercially available PSC maintenance medium, for example, the aforementioned Essential 8 medium (Es8, Thermo Fisher Scientific), SPM (Stem-Partner (registered trade mark) Human iPS/ES cells medium, KYOKUTO PHARMACEUTICAL INDUSTRIAL CO., LTD.), ReproFF2 medium (RFF2, REPROCELL), StemPro34 (invitrogen), RPMI-base medium, mTeSR1 (STEMCELL Technologies) and the like can also be used. In this step, Es8, SPM and the like are preferably used as a maintenance medium to predict and select a PSC clone that shows a differentiation potential in response to a differentiation stimulus. When cultured in a medium that confers differentiation resistance to PSC, such as RFF2 medium, PSC loses the ability to differentiate in response to differentiation stimulus. However, such differentiation resistance is not an inherent property of PSC clone, and differentiation potential may sometimes be reversibly recovered by changing the culture conditions. Therefore, when verifying the quality of PSC clone (whether differentiation resistance is an inherent property of the clone or reversible depending on the culture conditions), it is efficient to culture PSC with a medium that has been confirmed to maintain the differentiation potential of PSC in the present invention, such as Es8, SPM and the like. The medium may contain serum or may be serum-free. Where necessary, as long as the differentiation potential is not affected, the medium may contain one or more serum replacements such as albumin, transferrin, Knockout Serum Replacement (KSR) (serum substitute for FBS during ES cell culture), N2 supplement (Invitrogen), B27 supplement (Invitrogen), fatty acid, insulin, collagen precursor, trace element, 2-mercaptoethanol (2 ME), thiolglycerol and the like, and may also contain one or more substances such as lipid, amino acid, L-glutamine, Glutamax (Invitrogen), non-essential amino acid, vitamin, growth factor, low-molecular-weight compound, antibiotic, antioxidant, pyruvic acid, buffering agent, inorganic salts and the like.

In the step of culturing PSC, PSC may be cultured in single cells. For example, PSC is made into single cells by pipetting, trypsin treatment, and the like (to be also referred to as “single cell suspension”), seeded on a dish coated with VTN-N, and cultured feeder cell-free, whereby PSC can be cultured in single cells. In the step of culturing PSC, PSC may be cultured in a small cell clump. For example, PSC can be cultured in a small cell clump by culturing PSC on feeder cells (to be also referred to as “Small Cell Clumps method”).

In the step of culturing PSC, the culture temperature is not limited to the following, and is, for example, about 30-40° C., preferably about 37° C., and the culture is performed in an atmosphere of CO₂-containing air. The CO₂ concentration is about 2-10%, preferably 5%.

In the step of culturing PSC, the medium can be changed during the culture period. The medium used for the medium change may be a medium having the same components as the medium before the medium change or a medium having different components. Preferably, a medium having the same components is used. The timing of the medium change is not particularly limited, but may be performed, for example, every day, every two days, every three days, every four days or every five days after starting culture with a fresh medium. In this step, the medium change is preferably performed every day.

Measurement of the expression level of CHD7 of hPSC can be performed using any RNA or protein measurement method known per se. For example, when measuring the expression of CHD7 at the RNA level, Northern hybridization, RT-PCR, digital PCR and the like can be performed using a nucleic acid (probe) that can hybridize with the CHD7 mRNA under stringent conditions, or an oligonucleotide set that can function as a primer that amplifies a part or all of the mRNA. The nucleic acid to be used as the probe may be DNA or RNA, or DNA/RNA chimera. Preferably, DNA is used. The nucleic acid may be double-stranded or single-stranded. In the case of double-stranded, it may be a double-stranded DNA, a double-stranded RNA or a DNA:RNA hybrid. The length of the nucleic acid is not particularly limited as long as it can specifically hybridize with the target mRNA, and is, for example, about 15 bases or more, preferably about 20 bases or more. The nucleic acid is preferably labeled with a labeling agent to enable detection and quantification of the target mRNA. As the labeling agent, for example, radioisotope, enzyme, fluorescent substance, luminescent substance, and the like are used. As the radioisotope, for example, [³²P], [³H], [¹⁴C] and the like are used. As the enzyme, a stable enzyme having high specific activity is preferable. For example, β-galactosidase, β-glucosidase, alkaline phosphatase, peroxidase, malate dehydrogenase and the like are used. As the fluorescent substance, for example, fluorescamine, fluorescein isothiocyanate and the like are used. As the luminescent substance, for example, luminol, luminol derivative, luciferin, lucigenin and the like are used. Furthermore, biotin-(strept)avidin can also be used for binding the probe and the labeling agent.

The oligonucleotide set to be used as a primer is not particularly limited as long as it can be annealed specifically to each of the base sequence of mRNA encoding CHD7 (sense strand) and a base sequence complementary thereto (antisense strand), and can amplify the DNA fragment sandwiched between them. For example, a set of oligonucleotides each designed to have a length of about 15 to about 100 bases, preferably about 15 to about 50 bases, and to amplify a DNA fragment of about 100 bp to several kbp can be mentioned. In particular, a primer set capable of specifically amplifying mRNA of CHD7 isoform 1 (NCBI database (GenBank accession number NM 017780.3) also referred to as CHD7L (base sequence is shown in SEQ ID NO: 1 and amino acid sequence is shown in SEQ ID NO: 2) and capable of annealing to a base sequence of mRNA encoding the amino acids 672 to 2620 of CHD7 isoform 1 and a base sequence complementary thereto can be preferably used.

To quantitatively analyze the gene expression of CHD7 by using a trace amount of RNA sample, it is preferable to use competitive RT-PCR, real-time RT-PCR or digital PCR analysis. When competitive RT-PCR is used, a nucleic acid that affords an amplification product that is amplified by the above-mentioned primer set and can be distinguished from the target DNA (for example, an amplification product having a different size from the target DNA, an amplification product showing a different migration pattern by a restriction enzyme treatment and the like) can be further contained in addition to the primer set. The competitor nucleic acid may be DNA or RNA. In the case of DNA, PCR may be performed by adding a competitor after synthesizing cDNA from an RNA sample by a reverse transcription reaction. In the case of RNA, RT-PCR can be performed by adding a competitor to an RNA sample from the beginning. In the latter case, the efficiency of the reverse transcription reaction is also taken into consideration, and thus the absolute amount of the original mRNA can be estimated.

On the other hand, since real-time RT-PCR can monitor the amount of PCR amplification in real time, electrophoresis is not necessary and CHD7 gene expression can be analyzed more quickly. Generally, monitoring is performed using various fluorescent reagents. Among these are reagents that emit fluorescence by binding to double-stranded DNA (intercalators) such as SYBR Green I, ethidium bromide and the like, a nucleic acid that can be used as the above-mentioned probe (note that, the nucleic acid hybridizes to the target nucleic acid in the amplification region), and has both ends modified with a fluorescent substance (e.g., FAM, HEX, TET, FITC etc.) and a quenching substance (e.g., TAMRA, DABCYL etc.), and the like.

Digital PCR analysis is an analysis method that absolutely measures the expression level of a target gene in a sample by synthesizing cDNA from the extracted mRNA, diluting and dispersing the cDNA in the water droplets of ultra-fine compartment or water-in-oil (W/O) emulsion so that 1 or 0 cDNA will be contained, performing PCR amplification, and directly measuring the expression level of the target gene in the sample by directly counting the number of micro-compartments with positive amplification signals and the number of water droplets. This method can be used particularly preferably. For digital PCR analysis, a commercially available digital PCR analyzer can be used, the QuantStudio 3D digital PCR system (trade name of Thermo Fisher Scientific), BioMark HD (trade name of Fluidigm) and the products of Bio-Rad Laboratories adopting Droplet Digital PCR method, and the like can be used, and analysis can be performed according to the instruction manuals and protocols of each device.

The nucleic acid used as the above-mentioned probe may be cDNA encoding CHD7 or a fragment thereof, or may be obtained by chemical synthesis using a commercially available DNA/RNA automatic synthesizer and the like, based on the base sequence information (e.g., in the case of human CHD7, reference can be made to the base sequence registered under accession number NM_017780.3 (SEQ ID NO: 1) in the NCBI database (GenBank)). The oligonucleotide set to be used as the above-mentioned primer can be obtained by chemically synthesizing the base sequence and a part of the complementary chain sequence thereof based on the above-mentioned base sequence information and using a commercially available DNA/RNA automatic synthesizer or the like.

On the other hand, when the expression of CHD7 is measured at the protein level, it can be performed using various immunological methods, for example, various immunoassays such as Western blotting, ELISA, RIA, FIA and the like by using an anti-CHD7 antibody. The anti-CHD7 antibody to be used may be either a polyclonal antibody or a monoclonal antibody, may be prepared by a well-known immunological method, or a commercially available antibody may also be used. The antibody includes not only a complete antibody molecule but also a fragment thereof, and examples thereof include Fab, F(ab′)2, ScFv, minibody and the like. For the detail of the above-mentioned immunological measurement methods, reference can be made to, for example, Hiroshi Irie ed., “Radioimmunoassay” (Kodansha, published 1974), Hiroshi Irie ed., “Supplementary volume of Radioimmunoassay” (Kodansha, published 1979), Eiji Ishikawa et al. ed., “enzyme immunoassay” (Igaku-Shoin, published 1978), Eiji Ishikawa et al. ed., “enzyme immunoassay” (2nd edition) (Igaku-Shoin, published 1979), Eiji Ishikawa et al. ed., “enzyme immunoassay” (third ed.) (Igaku-Shoin, published 1987), “Methods in ENZYMOLOGY” Vol. 70 (Immunochemical Techniques (Part A)), ibidem Vol. 73 (Immunochemical Techniques (Part B)), ibidem Vol. 74 (Immunochemical Techniques (Part C)), ibidem Vol. 84 (Immunochemical Techniques (Part D: Selected Immunoassays)), ibidem Vol. 92 (Immunochemical Techniques (Part E: Monoclonal Antibodies and General Immunoassay Methods)), ibidem Vol. 121 (Immunochemical Techniques (Part I: Hybridoma Technology and Monoclonal Antibodies)) (all above published by Academic Press) and the like.

When the expression of CHD7 is measured at the protein level by sandwich ELISA, the selection of the combination of capture antibody and detection antibody is not particularly limited as long as CHD7 can be detected. Specifically, for example, a monoclonal antibody (mouse IgG1) is obtained using an antigen, a polypeptide in which Ala 263-Gln 457 of human CHD7 (SEQ ID NO: 2) are expressed in Escherichia coli, purifying with Protein A or Protein G, and can be used as a capture antibody. Specifically, for example, anti-human CHD7 rabbit IgG is obtained by immunizing a rabbit with Gly 25-Met 200 of human CHD7 (SEQ ID NO: 2) expressed in Escherichia coli, affinity purified using the antigen, and can be used as a primary antibody.

For the measurement of the expression level of CHD7, the expression level of isoform of CHD7 can be measured. Examples of the isoform include isoform 1 (NCBI database (GenBank accession number NM_017780.3) (base sequence is shown in SEQ ID NO: 1 and amino acid sequence is shown in SEQ ID NO: 2)), isoform 2 (i.q., accession number NM_001316690.1 (base sequence is shown in SEQ ID NO: 3 and amino acid sequence is shown in SEQ ID NO: 4)), isoform X4 (i.q., accession number XM_011517560.2 (base sequence is shown in SEQ ID NO: 5 and amino acid sequence is shown in SEQ ID NO: 6)) and the like. Preferred is isoform 1.

Using any of the above-mentioned methods or other method known per se, the expression level of CHD7 of PSC is measured, and the measurement value is compared with the threshold value of CHD7 expression level necessary for PSC to show a differentiation potential in response to a differentiation stimulus. The threshold value varies depending on the measurement method of the CHD7 expression level to be used. When, for example, measuring the expression level of CHD7 gene by quantitative digital PCR analysis is, for example, not less than 2710 copies in 5 ng of the total RNA. When cultured in single cells without feeder cell on a dish coated with extracellular substrate, it is unlimitatively, for example, not less than 1502 or not less than 1500 copies in 5 ng of the total RNA. When hPSC is ESC and cultured with feeder cells by the Small Cell Clumps method, it is unlimitatively, for example, not less than 2710 copies or not less than 2120 copies; when cultured in single cells without feeder cell on a dish coated with extracellular substrate, it is unlimitatively, for example, not less than 3280 copies; when hPSC is iPSC and cultured with feeder cells by the Small Cell Clumps method, it is unlimitatively, for example, not less than 3080 copies or not less than 2280 copies; and when cultured in single cells without feeder cell on a dish coated with extracellular substrate, it is unlimitatively, for example, not less than 1502 copies or not less than 1500 copies.

When the expression level of CHD7 protein is measured, the threshold value is, for example, not less than 2.0 times, not less than 3.0 times, not less than 4.0 times, not less than 5.0 times, not less than 6.0 times, not less than 7.0 times, not less than 7.7 times, not less than 8.0 times, not less than 8.5 times, not less than 9.0 times, not less than 9.2 times, not less than 10 times, not less than 10.2 times or not less than 10.3 times, compared to CHD7 protein concentration of human pluripotent stem cell showing differentiation resistance. Examples of the human pluripotent stem cell showing differentiation resistance include human pluripotent stem cell cultured for not less than 5 passages with ReproFF2 medium. Furthermore, when similar to the above-mentioned measurement, the threshold value is, for example, not more than 90.3%, not more than 90.2%, not more than 90%, not more than 89.1%, not more than 88.3%, not more than 87.0%, not more than 85.8%, not more than 85%, not more than 83.4%, not more than 80.2%, not more than 80%, not more than 75%, not more than 70%, not more than 50%, compared to CHD7 protein concentration of human pluripotent stem cell showing normal differentiation potential. Examples of the human pluripotent stem cell showing normal differentiation potential include human pluripotent stem cell cultured for not less than 5 passages with Es8 or SPM medium.

For example, the threshold value may be determined in consideration of the expression level of CHD7 gene measured by quantitative digital PCR analysis. Examples of the threshold value in consideration of the expression level of CHD7 gene may be not less than 2 times, not less than 3 times, not less than 4 times, not less than 5 times and not less than 10 times, compared to CHD7 protein concentration of human pluripotent stem cell showing differentiation resistance. Examples of the human pluripotent stem cell showing differentiation resistance may be human pluripotent stem cell cultured for not less than 5 passages with ReproFF2 medium. Furthermore, when similar to the above-mentioned case, the threshold value is, for example, not more than 50%, not more than 70%, not more than 75%, not more than 80% or not more than 90%, compared to CHD7 protein concentration of human pluripotent stem cell showing normal differentiation potential. Examples of the human pluripotent stem cell showing normal differentiation potential may be human pluripotent stem cell cultured for not less than 5 passages with Es8 or SPM medium.

The CHD7 protein concentration may be a value obtained by directly measuring the CHD7 protein concentration of the lysate of the cultured cells, or may be a value relative to the lysate of the cultured cells as the standard. The cultured cell may be a stem cell showing differentiation resistance or a stem cell showing normal differentiation potential, and the lysate may or may not be concentrated.

Even when other measurement method is used, PSC clone (e.g., hESC clones such as H1, H9, KhES1 and the like) confirmed by EB formation assay or cytokine-induced differentiation assay to show differentiation potential by culturing with Es8 or SPM medium is cultured with Es8 or SPM, specifically, for example, when cultured in single cells without feeder cell on a dish coated with extracellular substrate, the expression level of CHD7 when preferably cultured for not less than 5 passages is measured by plural experiments and, for example, the maximum value or mean of the measurement value can be determined as a threshold value.

As a result of comparison, when the expression level of CHD7 is not less than the threshold value of CHD7 expression level necessary for PSC to show a differentiation potential in response to a differentiation stimulus, the PSC shows a differentiation potential in response to a differentiation stimulus. Therefore, it can be predicted that there is no risk of remaining undifferentiated cells (which cause tumorigenesis) when differentiation is induced or that the cells have a low risk. Conversely, if the expression level of CHD7 is less than the threshold value, the PSC shows differentiation resistance to differentiation stimulus. Therefore, when differentiation is induced, it can be predicted that there is a risk of remaining undifferentiated cells (which cause tumorigenesis) or that the cells have a high risk. As a result of the prediction, if the risk remains or the cells have a high risk, the differentiation resistance can be reduced or eliminated by introducing a nucleic acid encoding CHD7 or changing the medium with one confirmed to be able to maintain the differentiation potential of PSC (e.g., Es8, SPM) and culturing for at least 5 passages. In addition, as a result of the prediction, if iPSC cultured together with feeder cells has a risk or the cells have a high risk, differentiation resistance can be reduced or eliminated by culturing in single cells without feeder cell.

As described above, whether or not PSC shows differentiation potential in response to differentiation stimulus is not necessarily an inherent property of PSC clone, but rather, highly probably alters depending on the PSC maintenance culture conditions. That is, even if the PSC clone is the same, when the culture conditions are different, it may differentiate in response to a differentiation stimulus (e.g., when cultured with Es8 or SPM), or it may often show differentiation resistance (when cultured with RFF2).

Therefore, the present invention also provides a method for evaluating a medium for a human pluripotent stem cell comprising measuring an expression level of CHD7 of PSC (hereinafter to be also referred to as the “evaluation method of the present invention”). Examples of the test medium include various media that can be used for maintenance culture of PSC prior to the step of measuring the above-mentioned expression level of CHD7.

As described in the below-mentioned Examples, 5 passages (about 15 days) are preferable for completely changing the epigenetic state of CHD7 gene in PSC by changing the medium to a different medium. Therefore, in the evaluation method of the present invention, PSC is preferably cultured with a test medium for 5 passages or more prior to the step of measuring the expression level of CHD7. The culture of PSC with the test medium can be performed by a method similar to that in the above-mentioned prediction method of the present invention.

The measurement of the expression level of CHD7 in the above-mentioned evaluation method of the present invention can also be performed by a method similar to that in the above-mentioned prediction method of the present invention. The obtained measurement value is compared with the threshold value of the CHD7 expression level which is necessary for PSC to show differentiation potential in response to a differentiation stimulus in the above-mentioned prediction method of the present invention. When, for example, measuring the expression level of CHD7 gene by quantitative digital PCR analysis, the threshold value is, for example, not less than 2710 copies in 5 ng of the total RNA. When cultured in single cells without feeder cell on a dish coated with extracellular substrate, it is unlimitatively, for example, not less than 1502 or not less than 1500 copies in 5 ng of the total RNA. When hPSC is ESC and cultured with feeder cells by the Small Cell Clumps method, it is unlimitatively, for example, not less than 2710 copies or not less than 2120 copies; when cultured in single cells without feeder cell on a dish coated with extracellular substrate, it is unlimitatively, for example, not less than 3280 copies; when hPSC is iPSC and cultured with feeder cells by the Small Cell Clumps method, it is unlimitatively, for example, not less than 3080 copies or not less than 2280 copies; and when cultured in single cells without feeder cell on a dish coated with extracellular substrate, it is unlimitatively, for example, not less than 1502 copies or not less than 1500 copies.

When the expression level of CHD7 protein is measured, the threshold value is, for example, not less than 2.0 times, not less than 3.0 times, not less than 4.0 times, not less than 5.0 times, not less than 6.0 times, not less than 7.0 times, not less than 7.7 times, not less than 8.0 times, not less than 8.5 times, not less than 9.0 times, not less than 9.2 times, not less than 10 times, not less than 10.2 times or not less than 10.3 times, compared to CHD7 protein concentration of human pluripotent stem cell showing differentiation resistance. Examples of the human pluripotent stem cell showing differentiation resistance include human pluripotent stem cell cultured for not less than 5 passages with ReproFF2 medium. Furthermore, when similar to the above-mentioned measurement, the threshold value is, for example, not more than 90.3%, not more than 90.2%, not more than 90%, not more than 89.1%, not more than 88.3%, not more than 87.0%, not more than 85.8%, not more than 85%, not more than 83.4%, not more than 80.2%, not more than 80%, not more than 75%, not more than 70%, not more than 50%, compared to CHD7 protein concentration of human pluripotent stem cell showing normal differentiation potential. Examples of the human pluripotent stem cell showing normal differentiation potential include human pluripotent stem cell cultured for not less than 5 passages with Es8 or SPM medium.

In addition, the threshold value may be determined, for example, in consideration of the expression level of CHD7 gene measured by quantitative digital PCR analysis. The threshold value in consideration of the expression level of CHD7 gene is, for example, not less than 2 times, not less than 3 times, not less than 4 times, not less than 5 times or not less than 10 times, compared to CHD7 protein concentration of human pluripotent stem cell showing differentiation resistance. Examples of the human pluripotent stem cell showing differentiation resistance include human pluripotent stem cell cultured for not less than 5 passages with ReproFF2 medium. Furthermore, when similar to the above-mentioned case, the threshold value is, for example, not more than 50%, not more than 70%, not more than 75%, not more than 80%, not more than 90%, compared to CHD7 protein concentration of human pluripotent stem cell showing normal differentiation potential. Examples of the human pluripotent stem cell showing normal differentiation potential include human pluripotent stem cell cultured for not less than 5 passages with Es8 or SPM medium.

The CHD7 protein concentration may be a value obtained by directly measuring the CHD7 protein concentration of the lysate of the cultured cells, or may be a value relative to the lysate of the cultured cells as the standard. The cultured cell may be a stem cell showing differentiation resistance or a stem cell showing normal differentiation potential, and the lysate may or may not be concentrated.

As a result of comparison, when the expression level of CHD7 is not less than the threshold value of CHD7 expression level necessary for hPSC to show a differentiation potential in response to a differentiation stimulus, the test medium can perform maintenance culture of PSC so that PSC shows a differentiation potential in response to a differentiation stimulus. Therefore, when differentiation is induced, it can be evaluated that there is no risk of leaving undifferentiated cells (which cause tumorigenesis) or that the medium has a low risk. Conversely, if the expression level of CHD7 is less than the threshold value, the test medium performs maintenance culture of PSC so that PSC shows a differentiation potential in response to a differentiation stimulus. Therefore, when differentiation is induced, it can be evaluated that there is a risk of leaving undifferentiated cells (which cause tumorigenesis) or that the medium has a high risk.

When maintenance culture of PSC is performed using the medium evaluated in the evaluation method of the present invention that the medium performs maintenance culture of PSC such that PSC shows a differentiation potential in response to a differentiation stimulus, the medium is changed to a medium evaluated by the same evaluation method to be able to perform maintenance culture of PSC such that PSC shows a differentiation potential in response to a differentiation stimulus, and PSC is cultured, preferably not less than 5 passages, whereby the PSC can be altered to show a differentiation potential in response to a differentiation stimulus.

As demonstrated in the below-mentioned Examples, spontaneous differentiation can be induced in PSC without applying a differentiation stimulus by introducing a nucleic acid encoding CHD7 into PSC instead of changing the medium with a medium capable of maintenance culture of PSC such that PSC shows a differentiation potential in response to a differentiation stimulus. For example, after transplanting hPSC-derived differentiation cell population, when an undifferentiated cell remains in the graft cells, the risk of tumorigenesis may be reduced by injecting a nucleic acid (mRNA, plasmid DNA etc.) encoding CHD7 into the transplantation site and introducing the nucleic acid into the remaining undifferentiated cells, thus causing spontaneous differentiation regardless of the environment of the transplantation site.

Therefore, the present invention also provides a differentiation-inducing factor for PSC, particularly hPSC, containing a nucleic acid encoding CHD7, and a method for inducing differentiation of PSC including introducing the nucleic acid into PSC showing differentiation resistance.

The present invention also provides a reagent for use in the above-mentioned prediction method of the present invention and the evaluation method of the present invention. The reagent contains a substance capable of detecting the expression of CHD7. Such substance is not particularly limited. For example, when the expression of CHD7 is detected at the RNA level, the nucleic acid and the like exemplified as the probe or primer in the above-mentioned prediction method of the present invention can be recited. When the expression of CHD7 is detected at the protein level, anti-CHD7 antibody and the like can be mentioned. The reagents can also be provided as a kit further containing various reagents necessary for carrying out the various methods for measuring the aforementioned expression level of CHD7.

While the present invention is further specifically explained in the following by showing Examples, the present invention is not at all limited by these Examples.

### EXAMPLE

### Material and Method

In the present specification, all the experiments conducted in this study used human ESCs and human iPSCs. The study was approved by the ethical committee of the Foundation for Biomedical Research and Innovation at Kobe (FBRI).

### (Cell Culture)

Any of human ESC lines KhES-1 (Riken BRC) (Navarro-Alvarez et al., Cell Transplant 2008; 17(1-2): 111-119) and H9 (WiCell Research Institute, Levenstein et al., Stem Cell; 24(3):568-74, 2006 March), and the human iPSC lines PFX #9 (Nishishita et al. PLoS One. 2012; 7(6): e38389), 201B7 (Riken RBC) and SHh #2 (Nishishita et al., PLoS One. 2012; 7(6)) were cultured with hPSCs culture media (Takenaka et al., PLoS ONE 2015; 10(6): e0129855) on mitomycin C-treated SNL76/7 cells (SIM strain embryonic fibroblast, ECACC; European Collection of Authenticated Cell Culture) or with Essential 8 medium (Es8, Thermo Fisher Scientific), (Chen et al., Nat Methods 2011; 8(5): 424-429), SPM (Stem-Partner (registered trade mark) Human iPS/ES cells medium) (Takenaka et al., PLoS ONE 2015; 10(6): e0129855) (Kyokuto Pharmaceutical Co. Ltd.) or RiproFF2 medium (RFF2, ReproCELL) on rhVitronectin-N (recombinant human Vitronectin-N) (Thermo Fisher Scientific)-coated dishes. Cells were passaged in clumps using Gentle Cell Dissociation Reagent (GCDR; Stem Cell Technologies) and split at a ratio of 1:3 for iPSCs or 1:3.5 for ESCs. Alternatively, cells were passaged by seeding a single-cell suspension using TrypLE Select (Life Technologies) (Takenaka et al., PLoS ONE 2015; 10(6): e0129855). Cells in single-cell suspensions were seeded at 3×10⁵ cells/well in 6-well plates when cultured with Es8 medium and 1×10⁵ cells/well with RFF2 medium. Cells were cultured in an atmosphere containing 5% CO₂ in an incubator (MCO-19AIC; Panasonic) at 37° C. Karyotypes of KhES-1, H9, and PFX #9 cells were examined by multicolor fluorescence in situ hybridization (mFISH) every 5 passages and by G-banding every 10 passages; PSCs with a normal karyotype were used in this study.

(siRNA Reagents and Transfection)

All reagents were purchased from Thermo Fisher Scientific, unless otherwise specified. Silencer Select Pre-designed human CHD7 siRNA (catalog No. 4392420; ID: s31142) and Silencer Select Negative Control No. 1 (catalog No. 4404021) were used for siCHD7 or control siRNA (mock) transfection experiments, respectively. Cells were transfected as follows.

ESCs were seeded at 2×10⁵ cells/well on VTN-N-coated 6-well plates and cultured with 4 mL of Es8, SPM or RFF2 medium. The following day, the medium was changed, and siCHD7 or control siRNA was transfected into the cells with Lipofectamine RNAiMAX in accordance with the manufacturer's instructions (transfection amount 50 pmol, 30 pmol or 10 pmol). Briefly with regard to introduction of 50 pmol siCHD7, cocktail A (4 μL Lipofectamine RNAiMAX Reagent and 150 μL Opti-MEM Medium) was mixed with cocktail B (1 μL of 50 μM siCHD7 (50 pmol) or control siRNA (50 pmol) and 150 μL Opti-MEM Medium), and the mixture was then incubated for 5 min at room temperature. The mixed cocktail (240 μL) was used for transfection of ESCs with siCHD7 (final concentration 10 nM) or control siRNA (final concentration 10 nM), and cells were incubated for 48 h. The transfection efficiency of the reagent was assessed by qRT-PCT for detection of CHD7 mRNA in the transfected cells at the designated time points.

(Synthetic mRNA Reagents and Transfection)

The T7 promoter and T7 terminator were fused into the 5′ and 3′ coding DNA sequences for CHD7 isoform 2 (NM_001316690.1 (SEQ ID NO: 3)), respectively and cloned into the pMX vector. Synthetic mRNA for CHD7 isoform 2 was then generated using an mMESSAGE mMACHINE T7 ULTRA Transcription Kit after digesting the pMX vector with SfiI. Synthetic mRNA covering both chromodomain and SNF2-like ATPase/helicase domain (CHD7 DN1) was generated by the same manner. The region of the SANT-SLIDE domain in CHD7 was determined based on the homology with the published CHD1 sequence (Ryan et al., Embo j 2011; 30(13):2596-2609), and synthetic mRNA (CHD7 DN2) was generated in the same manner. The quantity of the resulting mRNAs was measured with an ND-1000 (Nano Drop). mRNA for enhanced green fluorescent protein (eGFP) obtained from the same vector backbone was used as the control (control mRNA, mock).

(mRNA Transfection)

ESCs (3×10⁵) were seeded in each well of a VTN-N-coated 6-well plate with RFF2 supplemented with 5 ng/mL fibroblast growth factor 2 (FGF2; Peprotech). mCHD7 or control mRNA (mock) was transfected into the cells with Lipofectamine Messenger MAX in accordance with the manufacturer's instructions. Briefly, cocktail A (3.75 μL Lipofectamine Messenger MAX transfection reagent and 125 μL Opti-MEM Medium) was incubated for 10 min at room temperature. Then, cocktail B (2.5 μg mCHD7 or control mRNA and 125 μL Opti-MEM Medium) was prepared and mixed with cocktail A, followed by incubated for 5 min at room temperature. The mixed cocktail (240 μL) was added to 4 mL of RFF2 supplemented with FGF2 to a final concentration of 5 ng/mL, and the cells were cultured with this medium for 24 h at 37° C. The transfection efficiency of the reagent was assessed by determining the expression of CHD7 mRNA by qRT-PCR in the transfected cells at the designated time points.

### (Methylation and GeneChip Analysis)

The methylation state of cultured ESCs or iPSCs was determined with the Infinium HumanMethylation450 BeadChip (Illumina). The methylation pattern in the promoter region was hierarchically clustered using Cluster 3.0 and visualized in Java Tree View. The methylation status of the respective genes in the promoter regions was assessed by a comparison with the gene expression signal obtained by GeneChip (Human Genome U133 Plus 2.0 Array; Affymetrix) array data to extract the candidate genes.

(EB Formation Assays after Transfection with siCHD7)

Cells cultured on VTN-N-coated wells with 4 mL of Es8 medium were washed once with PBS (−) 48 h after transfection with either siCHD7 (50 pmol) or control siRNA (mock). Cells were then scraped with a cell scraper (Iwaki), dissociated by pipetting, transferred into a low-attachment 6-well plate (Corning), and cultured with Essential 6 medium (Es6) with 10 mM ROCK inhibitor (Y-27632, Wako) for 1 day and with Es6 medium alone for 13 days for EB formation. Medium was changed every 2 days. The numbers and morphologies of EBs were observed microscopically (Olympus IX71; Olympus). Gene expression for CHD7 was determined by qRT-PCR and gene expression profiles were determined by TaqMan (registered trade mark) Scorecard Panel (A15870) using a qRT-PCR device (QuantStudio 12 K Flex). Primer sequences for CHD7 are shown in Table 1.

TABLE 1

| | | | |
|---|---|---|---|
| Gene | F/R | seq (5′-3′) | SEQ ID NO |

primer

| | | | |
|---|---|---|---|
| POU5F1 | F | GAA ACC CAC ACT GCA GCA GA | SEQ ID NO: 7 |
| POU5F1 | R | TCG CTT GCC CTT CTG GCG | SEQ ID 10: 8 |
| SOX2 | F | GGG AAA TGG GAG GGG TGC AAA AGA GG | SEQ ID NO: 9 |
| SOX2 | R | TTG CGT GAG TGT GGA TGG GAT TGG TG | SEQ ID NO: 10 |
| NANOG | F | CTC AGC TAC AAA CAG GTG AAG AC | SEQ ID NO: 11 |
| NANOG | R | TCC CTG GTG GTA GGA AGA GTA AA | SEQ ID NO: 12 |
| PF300 | F | TTG AAT GTA CAG AGT GCG GAA GA | SEQ ID NO: 13 |
| PE300 | R | AAC AGC CAT CAC AGA CGA ATG C | SEQ ID NO: 14 |
| CHD7 | F | GGT TCC CAC ACT CGT GCA TA | SEQ ID NO: 15 |
| CHD7 | R | TGC GCC TCG GGA CAG A | SEQ ID NO: 16 |
| CHD7 isoform 2 | F | CCC ATG AAA GCA ATG AGT AAT CC | SEQ ID NO: 17 |
| CHD7 iSoform 2 | R | TCC ATT GGT ATC CCA GCA CTT C | SEQ ID NO: 18 |
| CHD7 Chromodomain | R | TGA TGG ACT TGG AAC ACA AAG TG | SEQ ID NO: 19 |
| CHD7 Chromodomain | R | TGA AGG GAA GCG ACT TGG TT | SEQ ID NO: 20 |
| CHD7 sant slid | F | CAA ACA TGG CTA TGA GAA GTA CAA CTC | SEQ ID NO: 21 |
| CHD7 sant slide | R | CCG ACT CGT TCC AGA AAG CA | SEQ ID NO: 22 |
| GAPDH | F | CCA CTC CTC CAC CTT TGA CG | SEQ ID NO: 23 |
| GAPDHH | R | ATG AGG TCC ACC ACC CTG TT | SEQ ID NO: 24 |

| | | |
|---|---|---|
| Refseq No. | Gene | Taqman probe |

Taq man PCR primer set 1

| | | |
|---|---|---|
| NM_017780 | CHD7 | Hs00215610_m1 |
| XM_011517553.2 | | |
| XM_011517555.2 | | |
| XM_017013613.1 | | |
| XM_011517560 | | |
| XM_011517554.2 | | |

| | | | | |
|---|---|---|---|---|
| Refseq No. | Gene | P/F/R | Seq (5′-3′) | SEQ ID NO |

Taq Man PCR primer set 2

| | | | | |
|---|---|---|---|---|
| NM_001316690 | CHD7 | P | TATGACTCAGAAACCGAAACAGAAACGACA | SEQ ID NO: 25 |
| | CHD7 | F | GCCCTTTCTAGAGAAACCAGTG | SEQ ID NO: 26 |
| | CHD7 | R | AGGCACCCTTTCTTCTCCTG | SEQ ID NO: 27 |

Taq man PCR primer set 3

| | | | | |
|---|---|---|---|---|
| NM_017780 | CHD7 | P | CACGGACGCTATAAACGCCAACTCACTG | SEQ ID NO: 28 |
| XM_011517553.2 | CHD7 | F | GAATCTGCTTGTCTATGGTTGGG | SEQ ID NO: 29 |
| XM_011517655.2 | GHD7 | R | AGGATGGTTCTGCAGATGGT | SEQ ID NO: 30 |
| XM_017013613.1 | | | | |
| XM_011517554.2 | | | | |

(EB Formation Assays after Transfection with mCHD7)

Cells cultured on VTN-N-coated wells and RFF2 supplemented with FGF2 to a final concentration of 5 ng/mL were washed once with PBS (−) 24 h after introduction of either mCHD7 or control mRNA. Then the cells were scraped with a cell scraper (Iwaki), dissociated by pipetting, transferred into a low-attachment 6-well plate (Corning), cultured with RFF2 containing 10 mM ROCK inhibitor (Y-27632, Wako) and not containing FGF2 for 1 day, and cultured with RFF2 not containing FGF2 to form EB while changing the medium every day. The number and morphology of EBs were observed microscopically (Olympus IX71, Olympus). Gene expression for CHD7 was determined by qRT-PCR and gene expression profiles were determined by TaqMan (registered trade mark) Scorecard Panel (A15870) using a qRT-PCR device (QuantStudio 12 K Flex).

### (Quantitative RT-PCR)

According to the manufacturer's instructions, total RNA was extracted using the RNeasy micro kit (74004, QIAGEN). To synthesize cDNA by using the QuantiTect Reverse Transcription Kit (205311, QIAGEN), 1 μg of total RNA was used. Using TaqMan (registered trade mark) hPSC Scorecard Panel (A15870), quantitative PCR (qPCR) for profiling gene expression relating to the three germ layers and self-proliferation was performed. Using SYBR (registered trade mark) Select Master Mix and StepOnePlus, cDNA was synthesized from 500 ng of total RNA by reverse transcription reaction. The reaction was carried out under the conditions of 95° C. for 10 min, and 40 cycles at 95° C. for 15 sec, at 60° C. for 1 min and at 72° C. for 15 sec. The primers used are listed in Table 1. Relative quantification was calculated using the 2^{-ΔΔCt} method after normalization with glyceraldehyde-3-phosphate dehydrogenase (GAPDH).

### (Determination of Copy Number of CHD7 Transcript)

The copy number of the CHD7 transcript was determined by a Droplet Digital PCR system (Bio-Rad Laboratories). Specifically, cDNA was generated from 5 ng of total RNA extracted from KhES-1 cultured with Es8 or RFF2 using a probe of TaqMan (registered trade mark) Gene Expression Assay (Hs00215010_m1, Thermo Fisher Scientific). An emulsion of the RT-PCR reaction mixture was generated using a QX100 system (Bio-Rad Laboratories) according to the instruction manual. Thereafter, cDNA was respectively amplified from Es8 culture and RFF2 culture using an Applied Biosystems GeneAmp 9700 Thermalcycler. Each reaction mixture was composed of a 20 μL solution containing 10 μL of ddPCR probe Supermix, 1000 nM primer, 250 nM probe and template cDNA. The reaction was performed under the conditions of a treatment at 95° C. for 10 min, after which 40 cycles of denaturation at 94° C. for 30 sec and elongation reaction at 53° C. for 60 sec, and finally, at 98° C. for 10 min. After the reaction, raw fluorescence data of each well was analyzed with software QuantaSoft ver. 1.2 (Bio-Rad Laboratories).

### (Western Blotting for Detection of CHD7)

Cell lysates for Western blotting were prepared 72 h after seeding. Proteins were extracted using Complete Lysis-M (Roche), supplemented with protease inhibitor cocktail tablets (Complete Mini; Roche). Polyclonal sheep IgG anti-CHD7 antibody (AF7350; R&D Systems) and rabbit anti-sheep IgG (H+L) antibody labeled with horseradish peroxidase were respectively used as primary antibody and secondary antibody. The signal was detected with Chemi-Lumi One Super reagents (Nacalai Tesque). Total protein was measured with a bicinchoninic acid total protein assay kit (Nacalai Tesque) prior to application to the lane.

### (Preparation of Standard and Sample for Sandwich ELISA of CHD7)

ESC was cultured under various conditions. Specifically, H9 was cultured for 17 passages with Es8 (P1), KhES-1 was cultured for 10 passages with Es8 (P2), and KhES-1 was cultured for 11 passages with RFF2 (N). All cells were cultured in single cells on a VTN-N coated dish without feeder cell. Each cultured ESC was lysed according to the protocol of cOmplete Lysys-M (Merck KGaA, product number: 04719956001), the protein concentration was measured, dispensed by 500 μL, and rapidly frozen in liquid nitrogen and stored at −80° C. until use. The total protein concentration of P1, P2 and N was 1.25 mg/mL, 1.27 mg/mL and 0.84 mg/mL respectively. H9 cells were cultured with Es8 or RFF2, lysed according to the protocol of cOmplete Lysys-M, then Amicon Ultra-4, PLTK Ultracel-PL membrane, 30 kDa (Amicon (registered trade mark) Ultra-4 PLTK Ultracel-PL membrane, 30 kDa, catalog number: UFC803024, Merck KGaA) were used to concentrate at 4000×g, 4° C. and the concentrated products were respectively used as a standard and a negative control (N2). The standard and the negative control (N2) were dispensed by 500 μL, rapidly frozen in liquid nitrogen, and stored at −80° C. until use. The total protein concentration of the standard and the negative control (N2) was respectively 4.06 mg/mL and 10.23 mg/mL.

### (Antibody for Sandwich ELISA)

For Sandwich ELISA, as a capture (solid phase) antibody, an antibody obtained by purifying, with Protein A or Protein G, a monoclonal antibody (mouse IgG1) obtained using, as an antigen, a polypeptide in which Ala 263-Gln 457 of human CHD7 (SEQ ID NO: 2) are expressed in Escherichia coli was used; as a primary antibody, anti-human CHD7 rabbit IgG obtained by immunizing the rabbit with Gly 25-Met 200 of human CHD7 (SEQ ID NO: 2) expressed in Escherichia coli and affinity purifying the rabbit with the antigen was used; and as the secondary antibody, anti-rabbit IgG-HRP (SouthernBiotech, 4090-05) was used.

### (Sandwich ELISA)

Sandwich ELISA was performed by the following method. To determine the optimal conditions for sandwich ELISA, the concentrations of the primary antibody and the secondary antibody were examined under the conditions shown in the following Table. 100 μL of the capture antibody solution diluted to 3 μg/mL with D-PBS(−) was added to the well of a 96-well plate (MaxiSorp (registered trade mark), Nunc, 44-2404-21), sealed with a plate seal, and stored at 4° C. overnight. Thereafter, the capture antibody solution was removed by decantation, 200 μL of D-PBS(−) containing 0.05% Tween (registered trade mark) 20 (hereinafter to be also referred to as “washing solution”) was added, and the mixture was gently stirred, allowed to stand for 5 min, removed and washed. This washing step was repeated 3 times. Blocking was performed by adding a blocking solution obtained by adding 1% BSA to the washing solution to the well after removal of the washing solution and incubating the mixture at room temperature for 1 hr. Then, the standard diluted with the blocking solution, the object sample, was added at 100 μL/well. To the Blank well was added the same amount of the blocking solution. A plate seal was adhered tightly to prevent evaporation of the solution and the mixture was incubated at 4° C. overnight. Thereafter, the well was washed three times with the washing solution, 100 μL of the primary antibody diluted to 1 μg/mL or 3 μg/mL with the blocking solution was added, and the mixture was incubated at room temperature for 1 hr. Thereafter, the primary antibody was removed and the well was washed 3 times with the washing solution. Then, 100 μL of the secondary antibody diluted 5000-fold or 10000-fold was added to the well and the mixture was incubated at room temperature for 1 hr. Thereafter, the mixture was washed 3 times with the washing solution, 100 μL of the substrate solution (TMB, ScyTek Laboratories, TM4500) was added, and the mixture was incubated for 20-30 min in the dark and 0.5 M H₂SO₄ was added at 100 μL/well to stop the reaction. The absorbance at 450 nm (Abs 450 nm) and 650 nm (reference wavelength, Abs 650 nm) was measured with a microplate reader wherein the CHD7 concentration of the stock solution of the standard was used as 1000 units, not less than 8 points in the 2-fold diluted dilution series or not less than 6 points in the 3-fold diluted dilution series from 500 U/mL were produced and measured.

TABLE 2

| Condition | Capture antibody | First Antibody | Secondary Antibody |
|---|---|---|---|
| F1/S5k | 3 μg/mL | 1 μg/mL | 1/5000 dilution |
| F1/S10k | 3 μg/mL | 1 μg/mL | 1/10000 dilution |
| F3/S5k | 3 μg/mL | 3 μg/mL | 1/5000 dilution |
| F3/S10k | 3 μg/mL | 3 μg/mL | 1/10000 dilution |

### (Calculation of CHD7 Protein Concentration)

The concentration of CHD7 was calculated by the following steps.

1) ΔAbs is calculated by the formula 1 from the measured absorbance:

ΔAbs= [symbol] Abs [symbol] 450 nm− [symbol] Abs [symbol] 650 nm (1)

2) Blank is subtracted from ΔAbs of each sample by the next formula 2 to calculate ΔAbs−blk:

ΔAbs−blk= [symbol] ΔAbs [symbol] Sample− [symbol] ΔAbs [symbol] Blank (2)

3) An analytical curve is drawn by the next formula 3 as a similar 4 parameter logistic curve:

f(x)=(a−d)/(1+(x/c)^{b})+d (3)

f(x): Abs−blk
x: CHD7 concentration (U/mL) after dilution
a: lower asymptote
b: inflection point inclination
c: inflection point
d: upper asymptote
4) ΔAbs−blk of the measurement sample is substituted into the equation of the analytical curve in the following formula 4 to calculate CHD7 concentration after dilution.

$$x = c \times \sqrt[b]{\frac{a - d}{f ( x ) - d} - 1} \qquad ( 4 )$$

5) CHD7 concentration after dilution of the measurement sample is multiplied by dilution factor to calculate CHD7 concentration before dilution.

### Results

### 1. Differentiation Potential of ESCs Alters by Changing Culture Conditions:

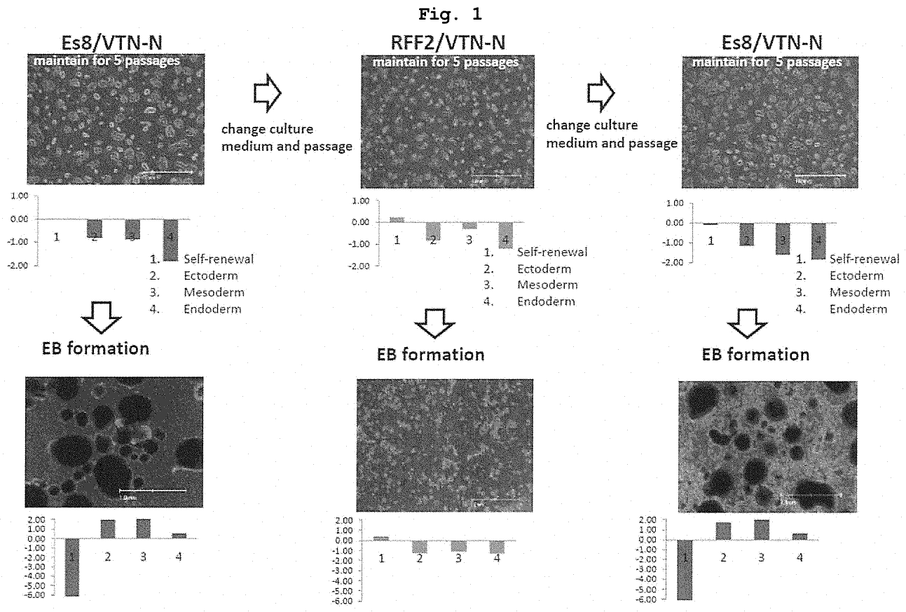

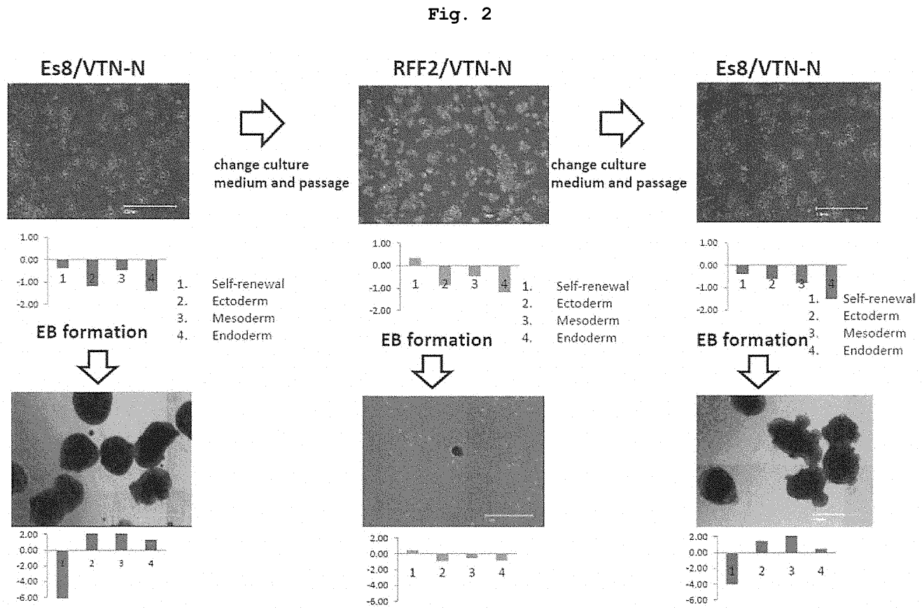

When KhES-1 cells showing a normal karyotype were cultured with Essential 8 medium (Es8) on an hrVitronectin-N(VTN-N)-coated dish, the KhES-1 cells maintained both self-proliferation ability and differentiation potential. KhES-1 cells lost differentiation potential after culturing for 5 passages with ReproFF2 (RFF2), but recovered the differentiation potential after culturing with Es8 (FIG. 1). While KhES-1 cells cultured with SPM (Takenaka et al., PLoS ONE 2015; 10(6): e0129855) maintained differentiation potential, the cells lost differentiation potential after culturing for 5 passages with RFF2. Furthermore, when PFX #9 iPSC (Nishishita et al., PLoS One 2012; 7(6): e38389) showing normal karyotype was cultured with Es8 and then with RFF2 on a VTN-N-coated dish, the same results were obtained (FIG. 2). These results indicate that the differentiation potential of PSC can reversibly alter depending on the culture conditions. It was considered that alterations in the epigenetic status of the cell relate to these responses.

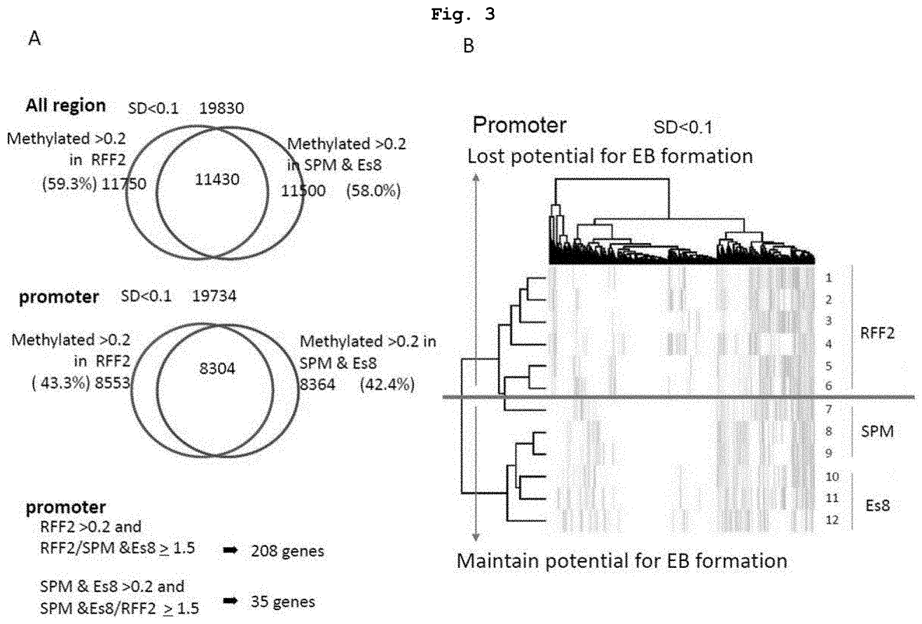

A comparison study of the methylation status of PSCs cultured with RFF2, Es8 or SPM was conducted using a methylation beads assay and characteristic methylation status that can lead to “loss of differentiation potential” of ESCs was identified. The comparative study results of the number of methylated genes in RFF2 culture and SPM and Es8 cultures are shown in FIG. 3A, and the clustering of methylation patterns in the promoter region is shown in FIG. 3B. Furthermore, the methylation status of promoters of major genes classified into self-proliferation, ectoderm, mesoderm, mesendoderm and endoderm displayed on scorecard panel (Thermo Fisher Scientific) was examined. The average methylation status in 6 PSC samples cultured with RFF2 (3 samples of iPSC and 3 samples of ESC) (RFF2), and 6 PSC samples cultured with SPM or Es8 (1 sample of iPSC and 2 samples of ESC cultured with SPM and 1 sample of iPS and 2 samples of ESC cultured with Es8) (SPM&Es8) is shown in Table (Table 3). All cells were maintained in an undifferentiated state, but there was no significant difference in the methylation pattern in the promoter region of these genes even when different medium was used. In Table 3, a numerical value of less than 0.2 means a low methylation status, a numerical value of 0.2 or more and less than 0.5 means a moderate methylation status, and a numerical value of 0.5 or more means a high methylation status.

TABLE 3

| GENE_SYMBOL | RFF2 | SPM&Es8 | Ratio |
|---|---|---|---|
| self-renewal | | | |
| KLF4 | 0.02 | 0.02 | 1.02 |
| KLF2 | 0.21 | 0.19 | 1.12 |
| TFOP2L1 | 0.06 | 0.06 | 0.97 |
| SOX2 | 0.02 | 0.02 | 0.94 |
| EP300(p300) | 0.04 | 0.04 | 0.85 |
| ZFP42(REX1) | 0.03 | 0.07 | 0.43 |
| NANOG | 0.36 | 023 | 1.58 |
| POU5F1 | 0.26 | 0.36 | 0.71 |
| HESX1 | 0.64 | 0.59 | 1.08 |
| LCK | 0.73 | 0.69 | 1.07 |
| DNMT3B | 0.69 | 0.71 | 0.97 |
| TRM22 | 0.88 | 0.89 | 0.98 |
| IDO1 | 0.92 | 0.93 | 0.99 |
| ectoderm | | | |
| COL2A1 | 0.04 | 0.04 | 0.98 |
| DRD4 | 0.04 | 0.06 | 0.78 |
| EN1 | 0.07 | 0.05 | 1.39 |
| LMX1A | 0.03 | 0.03 | 1.09 |
| NR2F1 | 0.04 | 0.03 | 1.29 |
| NR2F2 | 0.08 | 0.06 | 1.32 |
| OLFM3 | 0.08 | 0.07 | 1.14 |
| PAPLN | 0.10 | 0.11 | 0.91 |
| PAX3 | 0.05 | 0.04 | 1.45 |
| PAX6 | 0.03 | 0.03 | 1.00 |
| POU4F1 | 0.03 | 0.03 | 1.08 |
| PRKCA | 0.03 | 0.03 | 0.97 |
| SDC2 | 0.04 | 0.03 | 1.17 |
| SOX1 | 0.06 | 0.04 | 1.47 |
| WNT1 | 0.11 | 0.09 | 1.27 |
| ZBTB16 | 0.06 | 0.05 | 1.22 |
| CDH9 | 0.95 | 0.25 | 1.42 |
| LMX1A | 0.84 | 0.83 | 1.01 |
| DMBX1 | 0.82 | 0.83 | 0.99 |
| TRPM8 | 0.89 | 0.89 | 1.01 |
| NOS2 | 0.89 | 0.90 | 1.00 |
| MYO3B | 0.92 | 0.93 | 0.99 |
| mesoderm | | | |
| ALOX15 | 0.12 | 0.11 | 1.10 |
| CDX2 | 0.03 | 0.02 | 1.14 |
| FOXF1 | 0.04 | 0.03 | 1.28 |
| HAND1 | 0.03 | 0.03 | 1.06 |
| HAND2 | 0.05 | 0.03 | 1.56 |
| HEY1 | 0.01 | 0.02 | 0.86 |
| IL6ST | 0.04 | 0.04 | 1,02 |
| NKX2-5 | 0.10 | 0.04 | 2.75 |
| PDGFRA | 0,04 | 0.03 | 1.09 |
| SNAI2 | 0.05 | 003 | 1.35 |
| TBX3 | 0.03 | 0.03 | 0.98 |
| RGS4 | 0.33 | 0.19 | 1.71 |
| HOPX | 0.22 | 0.22 | 1.02 |
| ESM1 | 0.79 | 0.62 | 0.97 |
| CDH5 | 0.85 | 0.84 | 1.02 |
| TM4SF1 | 0.85 | 0.86 | 0.99 |
| PLVAP | 0.87 | 0.90 | 0.98 |
| ABCA4 | 0.89 | 0.90 | 0.98 |
| FCN3 | 0.90 | 0.91 | 0.98 |
| BMP10 | 0.92 | 0.92 | 1.00 |
| COLEC10 | 0.94 | 0.94 | 1.00 |
| mesendoderm | | | |
| FGF4 | 0.05 | 0.05 | 0.89 |
| NPPB | 0.14 | 0.09 | 1.44 |
| PTHLH | 0.03 | 0.03 | 1.18 |
| T | 0.03 | 0.03 | 1.18 |
| GDF3 | 0.80 | 0.83 | 0.97 |
| NR5A2 | 0.91 | 0.92 | 0.99 |
| endoderm | | | |
| CABP7 | 0.07 | 0.08 | 0.85 |
| CLDN1 | 0.03 | 0.03 | 1.01 |
| CPLX2 | 0.13 | 0.10 | 1.29 |
| EOMES | 0.07 | 0.04 | 1.66 |
| FOXA1 | 0.03 | 0.03 | 1.06 |
| FOXA2 | 0.04 | 0.04 | 0.98 |
| GATA4 | 0.04 | 0.03 | 1.13 |
| GATA6 | 0.02 | 0.02 | 0.91 |
| HHEX | 0.03 | 0.04 | 0.86 |
| HMP19 | 0.09 | 0.07 | 1.38 |
| HNF1B | 0.03 | 0.03 | 1.22 |
| KLF5 | 0.03 | 0.03 | 1.04 |
| NODAL | 0.06 | 0.07 | 0.83 |
| PHOX2B | 0.09 | 0.05 | 1.75 |
| POU3F3 | 0.03 | 0.03 | 1.04 |
| PRDM1 | 0.03 | 0.03 | 0.94 |
| SOX17 | 0.04 | 0.03 | 1 35 |
| ELAVL3 | 0.25 | 0.21 | 1.16 |
| SST | 0.42 | 0.40 | 1.05 |
| LEFT Y1 | 0.70 | 0.74 | 0.96 |
| FOXP2 | 0.83 | 0.78 | 1.06 |
| LEFTY2 | 0.84 | 0.81 | 1.03 |
| HNF4A | 0.91 | 0.90 | 1.01 |
| CDH20 | 0.91 | 0.93 | 0.98 |
| AFP | 0.92 | 0.94 | 0.98 |

Then, using GeneChip data (Affymetrix) of the same sample, a gene that showed high methylation status and low gene expression in RFF2 culture, and a gene that showed low methylation status and high gene expression in SPM or Es8 culture were investigated. By principal component analysis (PCA) and GeneChip analysis, four candidates were identified as methylation sites in the promoter region of candidate genes that could be markers for loss of differentiation potential (Table 4). Among the identified genes, chromodomain helicase DNA binding protein 7 (CHD7) was examined noting the function thereof.

TABLE 4

| | | Methylation | Gene expression |
|---|---|---|---|
| | | status | signal |
| | Description | RFF2/SPM/Es8 | RFF2/SPM/Es8 |
| DISP1 | Dispatched homologue 1 | 0.45/0.26/0.18 | 148/559/500 |
| RIMS3 | Regulating Synaptic | 0.33/0.21/0.17 | 566/1132/1369 |
| | membrane exocytosis 3 | | |
| NTS | neurotensin | 0.38/0.26/0.23 | 380/512/1721 |
| CHD7 | Chromodomain Helicase | 0.25/0.17/0.13 | 688/2553/3844 |
| | DNA binding Protein 7 | | |

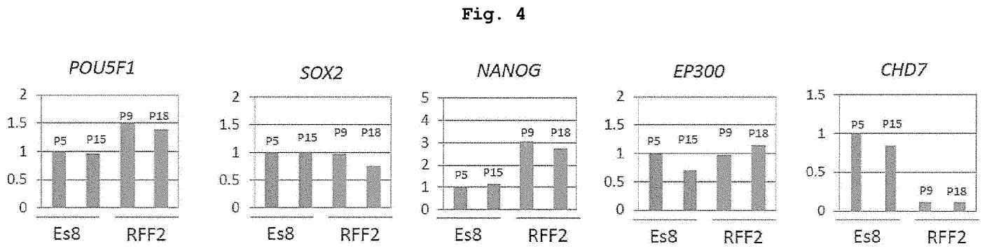

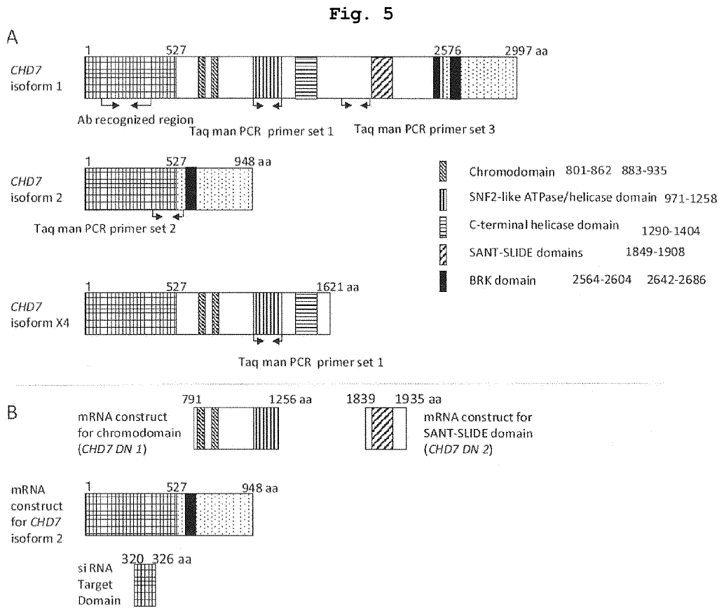

The effect of cell culture with RFF2 on the expression of self-proliferation-related genes such as NANOG, POU5F1, SOX2, EP300 and the like was determine using quantitative reverse transcription polymerase chain reaction (qRT-PCR). As a result, there was no significant effect on the expression level of self-proliferation-related genes. However, it was demonstrated by qRT-PCR that when primers were designed to target the 3′ non-translated region of CHD7 for detection of all CHD7 isoforms, the expression of the CHD7 gene was significantly suppressed by culturing ESCs with RFF2 (FIG. 4). The structures of isoform 1, isoform 2 and isoform X4 which are the three major isoforms of CHD7 (Schnetz et al., Genome Res 2009; 19(4): 590-601, Colin et al., BMC Res Notes 2010; 3: 252) and the positions of the primers are shown in FIG. 5A.

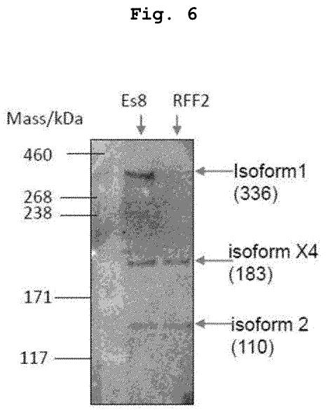

Next, the expression of three CHD7 isoforms was examined by Western blotting. As a result, CHD7 isoform 2 in cell lysate derived from Es8 and RFF2 cultures, and CHD7 isoform X4 in the both cell lysates were respectively detected at the same level by antibody recognizing the N-terminal of CHD7. However, CHD7 isoform 1 was not clearly detected in cell lysate derived from RFF2 culture (FIG. 6). The signal intensity of the CHD7 isoform cannot be compared and evaluated correctly by Western blotting when the molecular size of the target protein is different.

Based on the above, each isoform was quantified by digital PCR using an isoform-specific TaqMan primer (FIG. 5A). The copy number of CHD7 isoform 1, isoform 2 or isoform X4 determined by digital PCR is shown (Table 5). For quantification, total RNA (5 ng) obtained from KhES-1 cells cultured with Es8 or RFF2 medium was used as a template. Using primer set 3, the copy number of isoform 1 in each RNA sample was calculated. The copy number of isoform X4 was determined by subtracting the number of copies generated by primer set 3 from the number of copies generated by primer set 1 (FIG. 5A). Consistent with the results of Western blotting, isoform 1 was found to be the major isoform and initial target affected by culture conditions. Based on the results of Western blotting, the copy number of isoform 2 was expected to be the same as or less than the copy number of isoform X4. However, Taq man primer set 2 sandwiching the spliced sequence did not function properly due to primer design issues. Structural analysis of the CHD7 isoforms showed that isoform 1 contains a regulatory region extending from 527 to 2576 amino acids in the middle of the protein. This region contains ATPase/DNA helicase domains, a chromosome binding domain, a DNA binding domain, and a BRK domain (Allen et al., J Mol Biol 2007; 371(5): 1135-1140, Colin et al., BMC Res Notes 2010; 3: 252, Ryan et al., Embo j 2011; 30(13): 2596-2609). Isoform 2 lacks regulatory regions of isoform 1. Isoform X4 (FIG. 5A), which is another splicing variant of isoform 1, also altered gene expression thereof depending on the culture conditions.

TABLE 5

| | copy number in 5 ng total RNA | | | |
|---|---|---|---|---|
| Isoform type | Es8 P8 | Et8 P10 | RFF2 P5 | RFF2 P10 |
| Isoform 1 | 6380 | 7460 | 688 | 698 |
| isoform X4 | 220 | 320 | 52 | 34 |

Since it was shown that differentiation potential is related to CHD7 expression, whether KhES-1 cells cultured with Es8 lose differentiation potential when the expression level of mCHD7 downregulates by introduction of siRNA (FIG. 5B) was investigated. In addition, it was investigated whether KhES-1 cells cultured with RFF2 are differentiated when CHD7 mRNA was introduced. However, due to the length of the translation region, a full-length CHD7 isoform 1 mRNA could not be designed. Instead, CHD7 isoform 2 mRNA lacking the regulatory region (FIG. 5B) was prepared and mRNA was introduced into KhES-1 cells cultured with RFF2. Furthermore, to inhibit the activity by being competitive with the functional region of CHD7 isoform 1, mRNA encoding a DNA binding domain (SANT-SLIDE domain) assumed to be a regulatory region and mRNA producing a dominant negative protein encoding a chromatin interaction domain (chromodomain) and SNF2-like ATPase/helicase domain were respectively designed and introduced into KhES-1 cells cultured with Es8.

### 2. Down-Regulation of CHD7 Perturbs Differentiation:

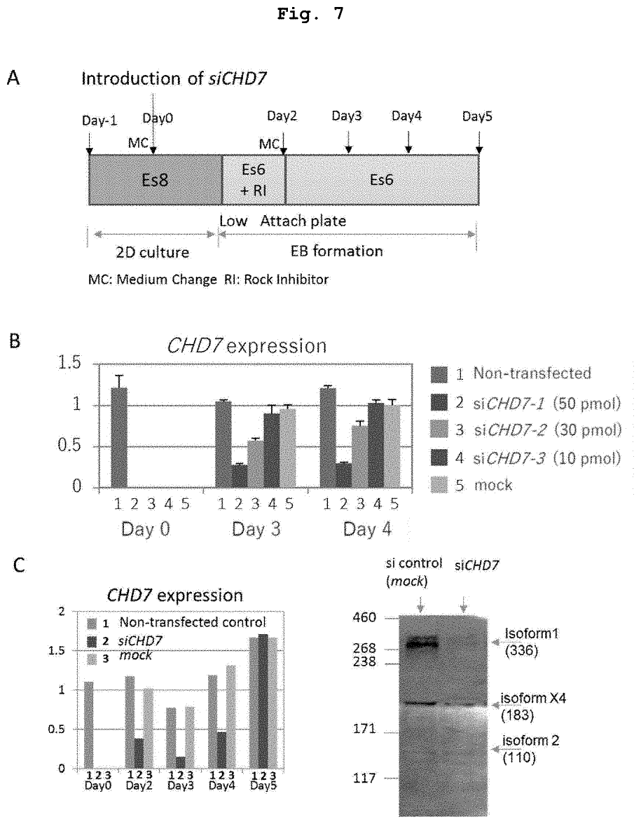

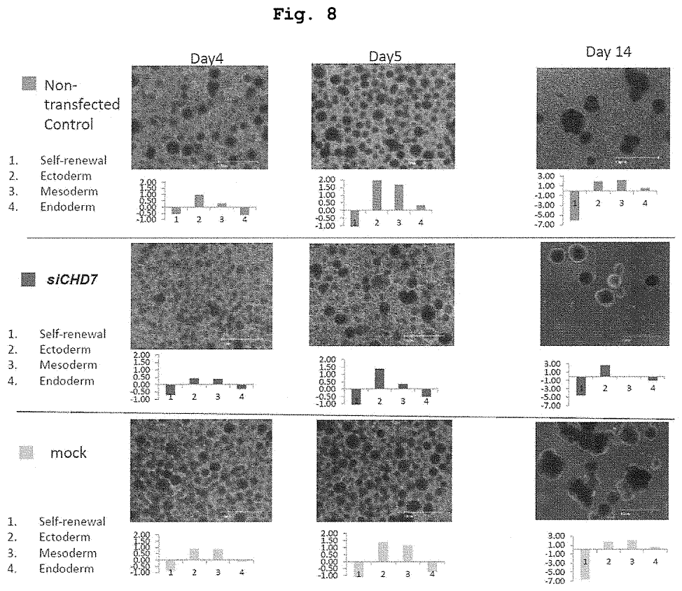

KhES-1 cells cultured with Es8 maintain differentiation potential and form EB. First, whether downregulation of CHD7 occurs in KhES-1 cells when siCHD7 is transfected was examined. 10 pmol, 30 pmol or 50 pmol of siCHD7 was transfected per well of a 6-well dish, and the expression level of CHD7 was examined. As a result, the expression level of CHD7 decreased as the introduced amount of siCHD7 increased, and the dependency of the introduction amount was observed (FIG. 7B). Next, the differentiation of siCHD7-transfected KhES-1 cells into three germ layers was examined. In EBs, perturbation of mesoderm differentiation was observed on day 5 and development of mesoderm and endoderm was suppressed to a moderate level on day 14 (FIG. 8). Downregulation of CHD7 mRNA with a single introduction of siCHD7 was not complete. A single introduction of siCHD7 failed to prevent initiation of ectoderm differentiation but perturbed development of mesoderm and endoderm after 14 days of culture. These data suggested that, to promote development of the three germ layers, the expression level of CHD7 needs to be maintained at a constant level throughout EB differentiation.

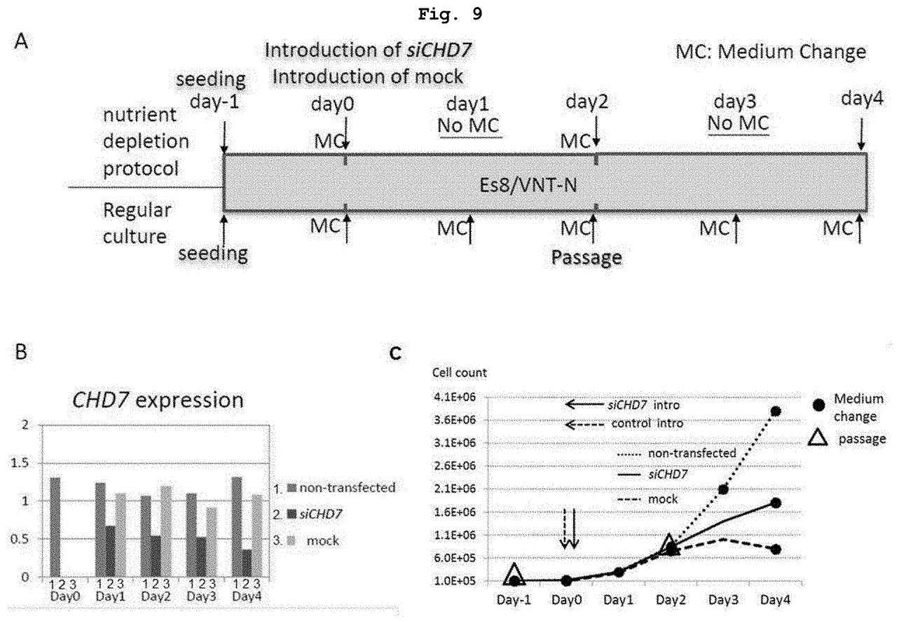

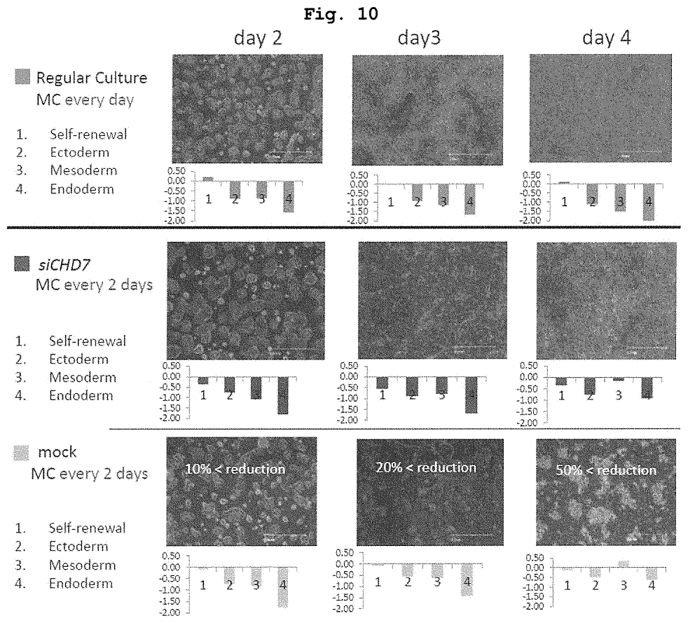

PSCs can be effectively cultured with high glucose (3.1 g/L) Es8 on a VTN-N-coated dish. When PSCs are cultured with nutrient-depleted Es8 (i.e., omitting daily medium change), differentiation of PSCs may be triggered (Vander Heiden et al., Science 2015; 324(5930): 1029-1033, Yanes et al., Nat Chem Biol 2010; 6(6): 411-417; Moussaieff et al., Cell Metab 2015; 21(3): 392-402). Mock-transfected KhES-1 cells cultured with nutrient-depleted Es8 were differentiated and were not maintained with Es8 on a VNT-N-coated dish. On the other hand, siCHD7-transfected KhES-1 cells remained on the VNT-N-coated dish 4 days after introduction (FIG. 9C), and exhibited a relatively undifferentiated genetic profile (FIG. 10). The cells did not proliferate in nutrient-depleted Es8, but downregulation of CHD7 expression via siCHD7 prevented differentiation caused by depletion of the nutrient (FIG. 10). Non-transfected KhES-1 cells with daily change of Es8 were used as normal culture control.

3. Introduction of CHD7 mRNA Induces Three Germ Layer Differentiation:

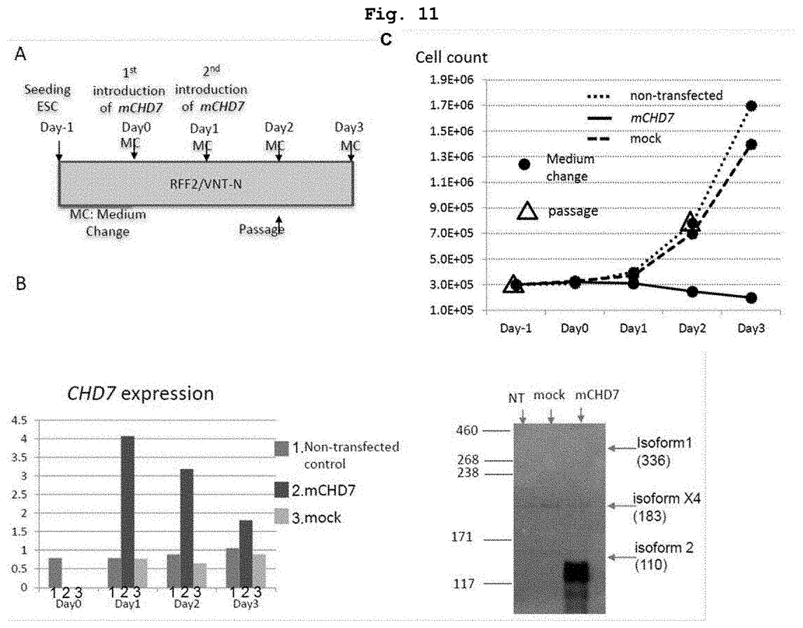

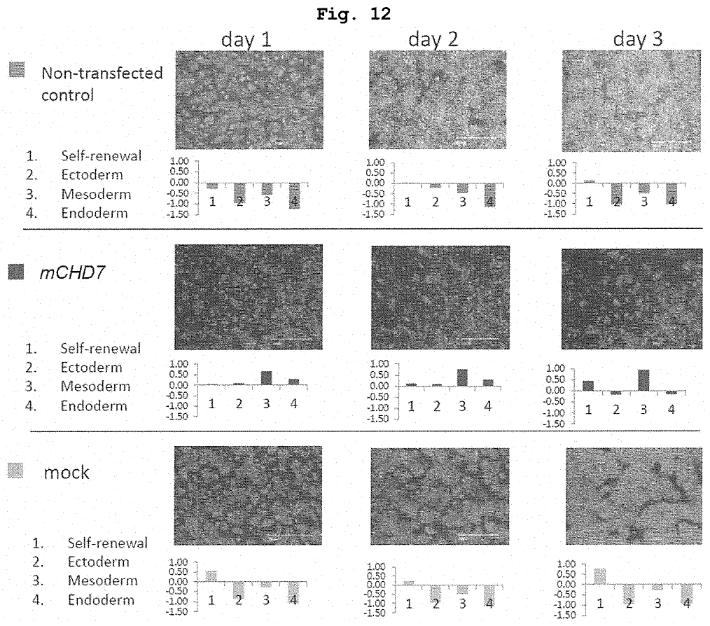

Introduction of CHD7 isoform 2 mRNA induced “spontaneous” differentiation of KhES-1 cells cultured with RFF2 without any additional differentiation stimulus (FIG. 12). KhES-1 cells cultured with RFF2 medium were transfected with CHD7 isoform 2, and the expression level of 94 genes in KhES-1 cells after 1, 2 and 3 days was determined by qRT-PCR scorecard panel (Table 6). In the Table, when fold change (fc) is not less than 2.0, it means upregulation, and when it is not more than 0.1, it means downregulation. The PSC culture system is designed to maintain undifferentiated cells rather than differentiated cells. When differentiated, KhES-1 cells could not be cultured on a VTN-N-coated dish, and the number of KhES-1 cells in the RFF2 culture decreased as the cells differentiated (FIG. 11C and FIG. 12). In particular, overexpression of CHD7 isoform 2 simultaneously induced three germ layer differentiation without following a continuous differentiation process.

TABLE 6-1

| | | KhES-1 RFF2 culture | | | | | | | | |
|---|---|---|---|---|---|---|---|---|---|---|
| | | Day1 | | | Day2 | | | Day3 | | |
| Target Name | Category | Nontrans | mock | mCHD7 | Nontrans | mock | mCHD7 | Nontrans | mock | mCHD7 |
| CXGL5 | Self-renewal | 12.60 | 10.05 | 7.18 | 8.51 | 6.78 | 5.12 | 6.31 | 6.68 | 4.87 |
| DNMT3B | | 1.23 | 1.0/ | 0.62 | 1.17 | 1.46 | 0.84 | 1.80 | 1.54 | 1.39 |
| HESX1 | | 0.41 | 0.28 | 0.24 | 0.26 | 0.21 | 0.21 | 0.37 | 0.39 | 0.32 |
| IDO1 | | 0.55 | 0.31 | 0.38 | 0.76 | 0.48 | 0.41 | 0.90 | 0.83 | 0.68 |
| LCK | | 1.28 | 0.99 | 1.07 | 1.70 | 1.54 | 0.92 | 2.01 | 1.76 | 1.10 |
| NANOG | | 3.09 | 2.94 | 2.19 | 4.67 | 3.17 | 2.42 | 3.92 | 3.24 | 2.62 |
| POU5F1 | | 0.98 | 0.64 | 0.55 | 1.80 | 1.30 | 1.35 | 1.63 | 1.30 | 0.77 |
| SOX2 | | 0.76 | 0.56 | 0.65 | 0.71 | 0.61 | 0.71 | 0.74 | 0.57 | 0.78 |
| TRIM22 | | 21.76 | 19.35 | 34.22 | 28.64 | 22.26 | 17.95 | 17.20 | 21.28 | 14.86 |
| COH9 | Ectoderm | 0.01 | 0.02 | 0.36 | 0.04 | 0.01 | 0.08 | 0.01 | 0.01 | 0.05 |
| COL2A1 | | 0.21 | 0.19 | 1.75 | 0.77 | 0.43 | 3.09 | 0.35 | 0.33 | 1.03 |
| DMBX1 | | 0.42 | 0.34 | 0.40 | 0.16 | 0.22 | 0.51 | 0.13 | 0.10 | 0.38 |
| DRD4 | | 0.22 | 0.24 | 0.50 | 0.32 | 0.22 | 0.46 | 0.33 | 0.33 | 0.56 |
| EN1 | | 0.65 | 1.23 | 69.19 | 0.87 | 2.96 | 8.41 | 0.44 | 0.36 | 2.43 |
| LMX1A | | 0.00 | 0.08 | 2.64 | 0.01 | 0.29 | 2.46 | 0.00 | 0.02 | 0.48 |
| MAP2 | | 7.24 | 11.63 | 10.18 | 14.79 | 16.34 | 14.96 | 2.66 | 3.86 | 5.35 |
| MY03B | | 0.43 | 0.29 | 0.35 | 0.76 | 2.36 | 0.32 | 0.54 | 0.81 | 0.65 |
| NOS2 | | 0.82 | 0.75 | 1.31 | 0.70 | 1.08 | 1.68 | 0.77 | 0.90 | 0.68 |
| NR2F1/NR2F2 | | 0.00 | 0.03 | 21.43 | 0.01 | 0.31 | 28.47 | 0.01 | 0.06 | 7.33 |
| NR2F2 | | 0.30 | 0.23 | 3.44 | 0.63 | 1.10 | 1.56 | 0.32 | 0.41 | 1.38 |
| OLFM3 | | 0.68 | 0.57 | 2.00 | 0.39 | 0.44 | 1.23 | 0.27 | 0.46 | 0.77 |
| PAPLN | | 0.19 | 0.21 | 0.41 | 0.44 | 0.34 | 0.43 | 0.36 | 0.39 | 0.35 |
| PAX3 | | 0.58 | 0.33 | 59.94 | 1.94 | 0.50 | 12.68 | 0.07 | 0.10 | 5.65 |
| PAX6 | | 0.13 | 0.18 | 16.25 | 0.11 | 0.13 | 10.65 | 0.04 | 0.11 | 2.76 |
| POU4F1 | | 0.40 | 0.04 | 42.21 | 0.04 | 0.10 | 33.30 | 0.49 | 0.32 | 7.77 |
| PRKGA | | 0.77 | 0.75 | 0.57 | 0.79 | 0.72 | 0.84 | 0.65 | 0.72 | 0.78 |
| SDC2 | | 29.34 | 19.66 | 20.10 | 26.51 | 18.98 | 18.81 | 17.11 | 16.78 | 15.36 |
| SOX1 | | 0.05 | 0.07 | 0.22 | 0.11 | 0.02 | 0.19 | 0.19 | 0.10 | 0.14 |
| TRPM8 | | 0.57 | 0.67 | 0.37 | 0.28 | 0.78 | 0.63 | 1.59 | 0.93 | 1.53 |
| WNT1 | | 1.73 | 4.36 | 3.56 | 1.45 | 1.49 | 4.66 | 2.25 | 1.82 | 10.01 |
| ZBTB16 | | 0.24 | 0.27 | 0.58 | 0.27 | 0.36 | 0.74 | 0.47 | 0.27 | 0.62 |
| ABCA4 | Mesoderm | 0.59 | 0.75 | 0.87 | 0.99 | 0.92 | 2.17 | 1.17 | 1.80 | 2.41 |
| ALOX15 | | 2.62 | 2.32 | 2.67 | 3.03 | 2.74 | 2.18 | 3.13 | 3.47 | 2.35 |
| BMP10 | | 1.29 | 1.24 | 3.39 | 3.85 | 2.46 | 3.14 | 0.32 | 1.29 | 0.05 |
| COH5 | | 6.21 | 4.44 | 3.01 | 4.59 | 11.32 | 7.23 | 1.09 | 3.76 | 8.08 |
| CDX2 | | 0.15 | 0.16 | 8.22 | 0.05 | 0.32 | 10.09 | 0.27 | 0.46 | 6.01 |
| COLEC10 | | 0.49 | 1.09 | 0.72 | 1.65 | 2.72 | 2.39 | 1.95 | 2.17 | 1.41 |
| ESM1 | | 1.83 | 1.58 | 1.74 | 0.03 | 0.56 | 0.51 | 2.82 | 2.68 | 3.67 |
| FCN3 | | 0.69 | 0.58 | 0.66 | 1.46 | 1.02 | 0.95 | 1.76 | 1.17 | 0.47 |
| FOXF1 | | 0.30 | 0.51 | 13.58 | 0.69 | 1.58 | 17.82 | 0.18 | 0.92 | 6.37 |
| HAND1 | | 0.18 | 0.26 | 1.57 | 0.14 | 0.70 | 1.66 | 0.10 | 0.89 | 5.67 |
| HAND2 | | 0.37 | 0.63 | 273.66 | 0.02 | 1.04 | 317.00 | 0.24 | 0.72 | 60.26 |
| HEY1 | | 0.74 | 0.65 | 1.33 | 0.59 | 0.77 | 1.01 | 0.44 | 0.51 | 0.72 |
| HOPX | | 1.90 | 0.40 | 15.67 | 2.09 | 0.50 | 2.38 | 0.28 | 0.44 | 1.46 |
| IL6ST | | 1.15 | 1.51 | 1.85 | 1.05 | 2.05 | 3.57 | 0.81 | 1.36 | 2.49 |
| NKX2-5 | | 2.60 | 2.56 | 33.32 | 2.65 | 3.16 | 25.85 | 2.11 | 2.34 | 6.08 |
| ODAM | | 0.96 | 1.71 | 15.69 | 1.30 | 1.48 | 4.54 | 0.53 | 1.40 | 1.87 |
| PDGFRA | | 1.25 | 0.84 | 0.57 | 0.70 | 0.84 | 0.36 | 0.51 | 0.81 | 0.73 |
| PLVAP | | 1.23 | 0.78 | 1.32 | 1.70 | 1.32 | 1.85 | 2.12 | 1.53 | 1.19 |
| RGS4 | | 0.77 | 0.32 | 1.53 | 0.30 | 0.37 | 3.24 | 1.27 | 1.48 | 2.02 |
| SNAI2 | | 0.60 | 0.72 | 0.73 | 0.39 | 0.59 | 0.75 | 0.32 | 0.46 | 0.97 |
| TBX3 | | 0.38 | 0.22 | 0.46 | 0.37 | 0.39 | 0.46 | 0.39 | 0.42 | 0.99 |
| TM4SF1 | | 0.38 | 0.22 | 0.46 | 0.37 | 0.39 | 0.46 | 0.39 | 0.42 | 0.99 |
| FGF4 | Mesendoderm | 10.49 | 5.56 | 5.04 | 12.41 | 11.18 | 5.41 | 12.77 | 16.21 | 18.31 |
| GDF3 | | 23.80 | 15.71 | 9.35 | 26.97 | 22.59 | 12.60 | 42.30 | 37.12 | 26.71 |
| NPPB | | 3.15 | 7.14 | 12.79 | 2.16 | 4.78 | 16.30 | 0.79 | 2.09 | 7.36 |
| NR5A2 | | 128.86 | 122.08 | 259.54 | 161.63 | 161.89 | 313.07 | 184.43 | 179.10 | 174.98 |
| PTHLH | | 1.02 | 1.24 | 45.09 | 0.52 | 1.36 | 32.69 | 0.28 | 0.73 | 9.52 |
| T | | 0.00 | 0.00 | 0.01 | 0.00 | 0.00 | 0.01 | 0.00 | 0.00 | 0.02 |
| AFP | Endoderm | 0.04 | 0.03 | 0.32 | 0.00 | 0.01 | 0.10 | 0.00 | 0.00 | 0.06 |
| CABP7 | | 2.02 | 2.05 | 5.18 | 2.70 | 2.48 | 3.55 | 3.66 | 5.14 | 8.92 |
| CDH20 | | 0.75 | 0.39 | 3.28 | 0.36 | 1.01 | 3.05 | 0.34 | 0.45 | 1.26 |
| CLDN1 | | 1.84 | 2.30 | 3.95 | 2.79 | 2.95 | 4.35 | 1.78 | 2.16 | 2.40 |
| CPLX2 | | 2.34 | 2.17 | 4.33 | 1.64 | 2.27 | 2.91 | 2.45 | 2.99 | 3.19 |
| ELAVL3 | | 0.81 | 0.88 | 3.33 | 3.67 | 2.07 | 2.33 | 2.45 | 1.70 | 0.85 |
| EOMES | | 0.02 | 0.07 | 13.59 | 0.05 | 0.05 | 5.22 | 0.03 | 0.03 | 1.94 |
| FOXA1 | | 0.10 | 0.07 | 21.37 | 0.07 | 0.19 | 14.07 | 0.02 | 0.09 | 4.44 |
| FOXA2 | | 0.01 | 0.03 | 0.11 | 0.04 | 0.05 | 0.27 | 0.04 | 0.05 | 0.29 |
| FOXP2 | | 0.24 | 0.12 | 3.96 | 0.22 | 0.32 | 7.37 | 0.12 | 0.16 | 2.35 |
| GATA4 | | 0.02 | 0.09 | 4.26 | 0.03 | 0.10 | 3.65 | 0.03 | 0.06 | 1.21 |
| GATA6 | | 0.10 | 0.27 | 2.67 | 0.08 | 0.48 | 2.40 | 0.03 | 0.25 | 1.28 |
| HHEX | | 0.10 | 0.11 | 4.36 | 0.16 | 0.12 | 2.62 | 0.13 | 0.21 | 1.01 |
| HMP19 | | 0.44 | 0.52 | 0.52 | 0.80 | 0.72 | 1.31 | 1.33 | 0.76 | 0.75 |
| HNF1B | | 0.02 | 0.02 | 0.36 | 0.03 | 0.05 | 0.32 | 0.01 | 0.05 | 0.22 |
| HNF4A | | 0.03 | 0.12 | 0.87 | 0.02 | 0.26 | 0.89 | 0.02 | 0.14 | 0.31 |
| KLF5 | | 4.02 | 6.91 | 19.30 | 4.14 | 5.08 | 11.28 | 1.97 | 2.96 | 6.25 |
| LEFTY1 | | 0.44 | 0.36 | 0.31 | 0.35 | 0.43 | 0.30 | 0.26 | 0.28 | 0.23 |
| LEFTY2 | | 0.26 | 0.23 | 0.14 | 0.11 | 0.19 | 0.23 | 0.05 | 0.09 | 0.14 |
| NODAL | | 1.11 | 1.06 | 0.95 | 1.19 | 1.57 | 1.40 | 0.71 | 0.70 | 1.15 |
| PHOX2B | | 0.03 | 0.03 | 2.55 | 0.03 | 0.03 | 1.73 | 0.03 | 0.04 | 0.40 |
| POU3F3 | | 0.05 | 0.08 | 3.46 | 0.05 | 0.06 | 1.53 | 0.08 | 0.07 | 0.44 |
| PRDM1 | | 0.21 | 0.17 | 1.74 | 0.31 | 0.49 | 3.66 | 0.34 | 0.44 | 1.70 |
| RXRG | | 0.05 | 0.98 | 2.07 | 0.01 | 0.86 | 6.17 | 0.05 | 0.35 | 2.02 |
| SOX17 | | 0.07 | 0.07 | 54.39 | 0.12 | 0.11 | 4.12 | 0.01 | 0.25 | 1.76 |
| SST | | 12.92 | 10.67 | 12.19 | 10.84 | 14.97 | 17.65 | 19.21 | 16.72 | 23.81 |

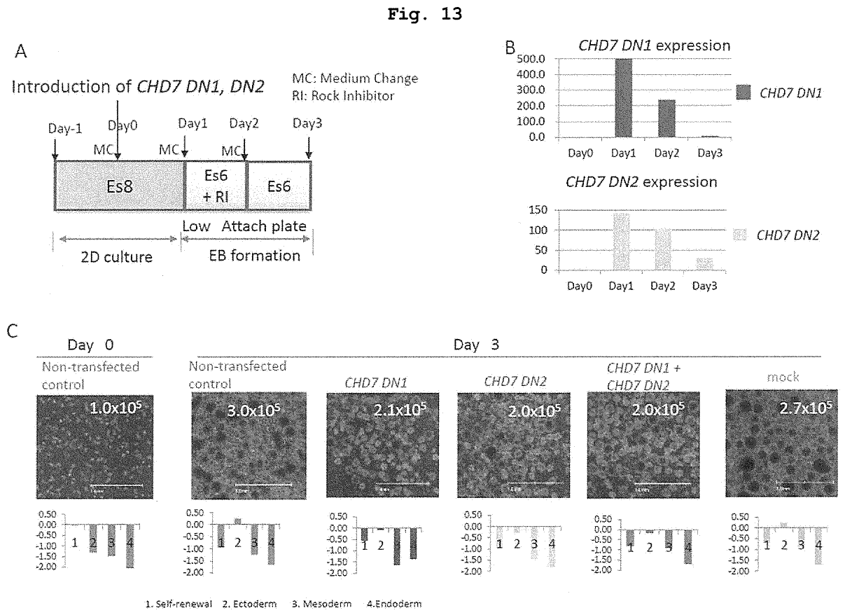

To inhibit or decrease CHD7 isoform 1 activity by being competitive with the functional region of CHD7 isoform 1, mRNA that generates a dominant negative protein covering chromodomain that recognizes the binding site of histone with a specific methylation status and the SNF2-like ATPase/helicase domain (CHD7 DN1) was introduced into KhES-1 cells (FIG. 5B and FIG. 13 A, B). By the introduction of CHD7 DN1, inhibition or depression of both differentiation potential and cell proliferation was observed in the EB formation assay (FIG. 13C). Furthermore, when mRNA that generates a dominant negative protein of SANT-SLID domain (CHD7 DN2), which is a putative DNA binding site of CHD7, was introduced into KhES-1 cells (FIG. 5B and FIG. 13 A, B), differentiation potential was also inhibited or depressed in the EB formation assay, thus showing decreased cell proliferation (FIG. 13C).

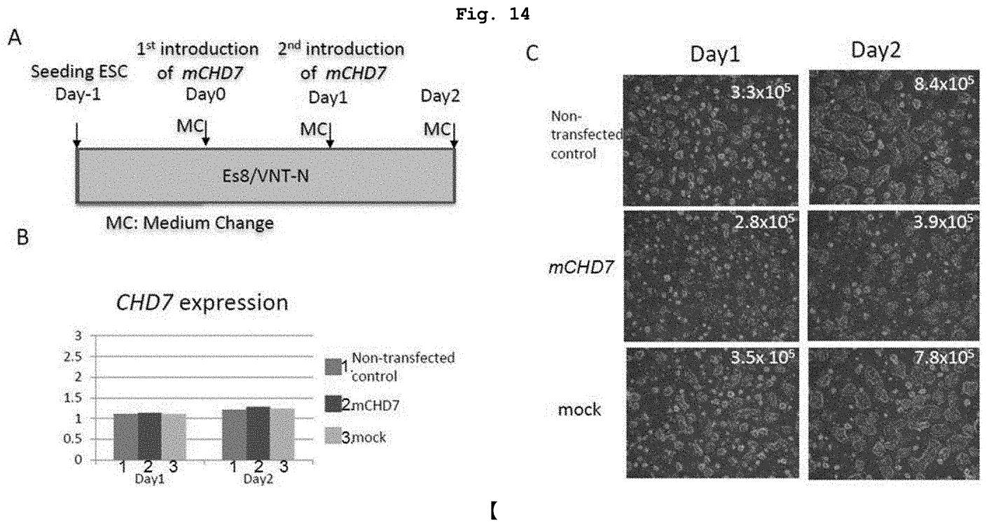

Introduction of CHD7 isoform 2 mRNA also suggested a possibility of the presence of the upper limit of CHD7 expression when ESCs are maintained in an undifferentiated state. The upper limit may sometimes vary depending on the culture conditions. Indeed, introduction of CHD7 isoform 2 mRNA into cultured KhES-1 cells with Es8 did not upregulate CHD7 isoform 2 in KhES-1 cells in Es8 culture (FIG. 14). It is considered that introduction of CHD7 isoform 2 mRNA into KhES-1 cells induces “spontaneous” differentiation of KhES-1 cells in Es8 and the Es8 culture system cannot support differentiated cells.

### 4. CHD7 Expression Level Regulates Proliferation Rate of Cells:

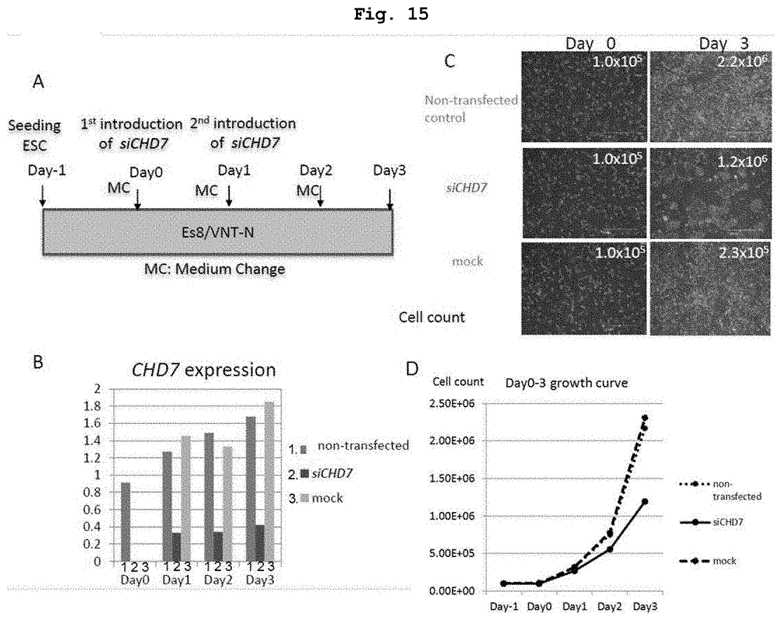

Another function of CHD7 is to support proliferation of ESCs. 1×10⁵ KhES-1 cells were seeded per well of a 6-well plate, the cells were cultured with Es8, and 8×10⁵ cells were harvested after 3 days. The proliferation rate of KhES-1 cells in RFES2 medium was ⅓ of that using Es8; however, when Es8 was used, KhES-1 cells were observed to expand 8 times after 3 days of culture. To examine whether the CHD7 expression level can modulate the proliferation rate of ESC in a concentration-dependent manner, CHD7 was downregulated by transfection of siCHD7 into KhES-1 cells, the cells were cultured with Es8 and the number of cells was counted (FIG. 15 A, B). As a result, the proliferation rate of KhES-1 cells was regulated by the expression level of CHD7 mRNA (FIG. 15 C, D). Furthermore, introduction of mRNA into KhES-1 cells that produces a dominant-negative protein of CHD7 covering the chromodomain and/or the SANT-SLIDE domain dramatically reduced the cell proliferation rate (FIG. 13).

### 5. Expression Level of CHD7 Mediates Differentiation Potential of PSCs:

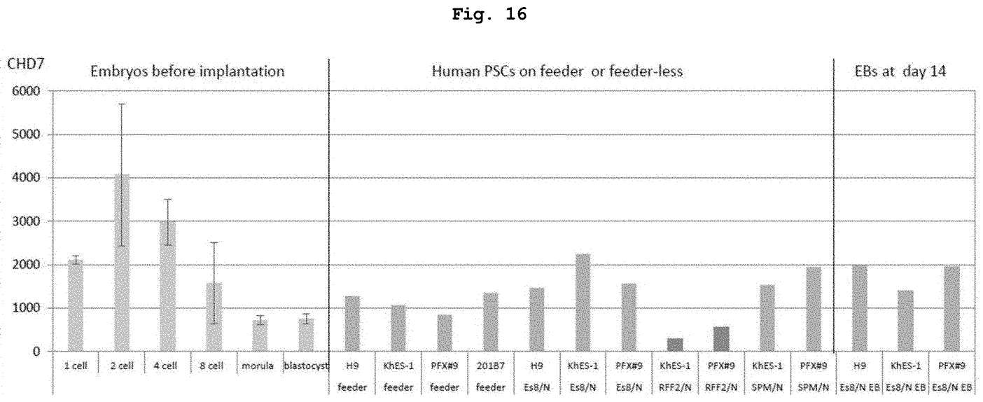

Whether the CHD7 mRNA level correlates with the differentiation potential of ESCs was examined by measuring the expression level of CHD7 mRNA of PSCs cultured under various conditions. First, using the GeneChip database, the expression level of CHD7 mRNA in embryo at various embryo formation stages before implantation, and the expression level of CHD7 mRNA of PSCs and EBs were examined (FIG. 16). CHD7 mRNA corresponding to isoform 1 was expressed at a relatively high level in the 2- and 4-cell stages and thereafter expressed at a low level in the morula stage and blastocyst stage (FIG. 16, “2 cell”, “4 cell”, “morula” and “blastocyst”). During the embryo formation, a fertilized egg is differentiated and proliferates. Embryos reach the blastocyst stage, which is composed of proliferation of the same cell mass without a developmental axis, which is called an inner cell mass. In the measurement of a GeneChip expression signal, PSCs showed a gene expression profile similar to the gene expression profile of mouse epiblast after implantation and various levels of CHD7 mRNA according to the culture conditions. PSCs showing low expression of CHD7 (FIG. 16, “KhES-1 RFF2/N” and “PFX #9 RFF2/N”) maintained a proliferation ability in an undifferentiated state but lost differentiation potential.

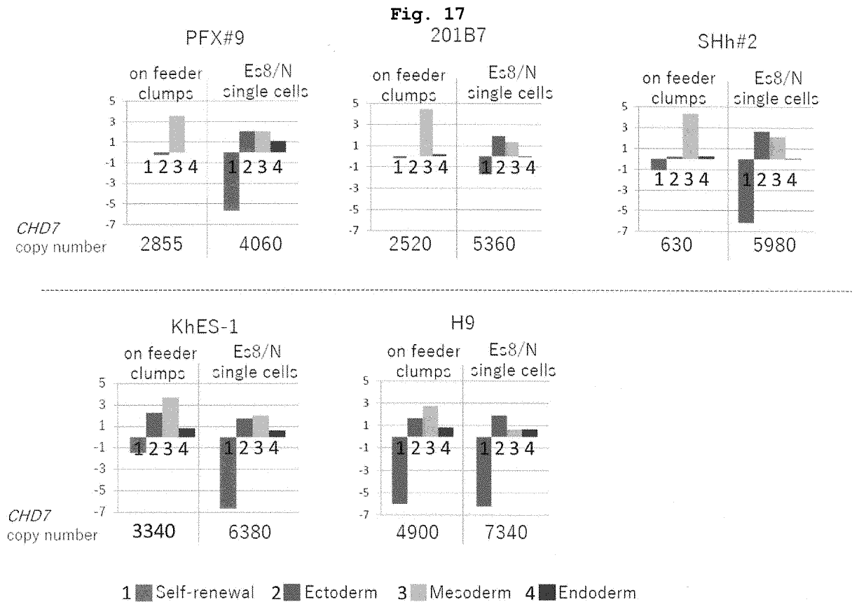

Since GeneChip expression signal may not be suitable for quantification, the copy number of CHD7 isoform 1 mRNA in 5 ng of the total RNA was quantified by digital PCR in various PSCs cultured under various conditions. Specifically, the cells used were H9 or KhES-1 (each ESC), or PFX #9, 201B7 or SHh #2 (each iPSC). The cells were cultured by the Small Cell Clumps method with hPSC medium on feeder cells. Alternatively, they were cultured in single cells on a VTN-N-coated dish with Es8, RFF2 or SPM. The copy number of CHD7 isoform 1 mRNA in 5 ng of the total RNA extracted from the cultured cells was determined by droplet digital PCR. PSCs cultured with RFF2 showed a low copy number of CHD7 isoform 1 in 5 ng of the total RNA and did not show differentiation potential; however, differentiation potential was confirmed in the cells cultured under other conditions. The copy number and passage numbers (P) thereof are shown in Table 7. The results of specifically analyzed differentiation potential of some of the cells cultured under these culture conditions are shown in FIG. 17.

TABLE 7

| | H9 | KhES-1:. | PFX#9 | 201B7 | SHh#2 | Passage Method |
|---|---|---|---|---|---|---|
| on feeder | 5220 (P29) | 2120 (P32) | 3080 (P25) | 4200 (P29) | 630 (P19) | Small Cell Clumps |
| | 5100 (P36) | 2338 (P32) | 3320 (P35) | 2280 (P35) | 542 (P24) | |
| | 4900 (P38) | 2710 (P37) | 2855 (P37) | 2520 (P40) | | |
| | | 3340 (P35) | | | | |
| Es8/VNT-N | 6560 (P5) | 9320 (P7) | 4620 (P5) | 6060 (P9) | 5120 (P5) | Single Cell |
| | 7340 (P17) | 7460 (P10) | 5740 (P11) | 5360 (P10) | 5980 (P6) | Suspension |
| | 8900 (P20) | 6380 (P10) | 4060 (P10) | | | |
| | | 6540 (P13) | 4260 (P16) | | | |
| SPM/VNT-N | 4520 (P5) | 4580 (P10) | 4540 (P15) | ND | | |
| | | 3940 (P10) | 4600 (P15) | | | |
| | | 6540 (P15) | | | | |
| RFF2/VNT-N | 352 (P11) | 688 (P5) | 652 (P20) | ND | 732 (P5) | |
| | 266 (P8) | 698 (P9) | 274 (P30) | | | |
| | | 490 (P10) | 246 (P45) | | | |

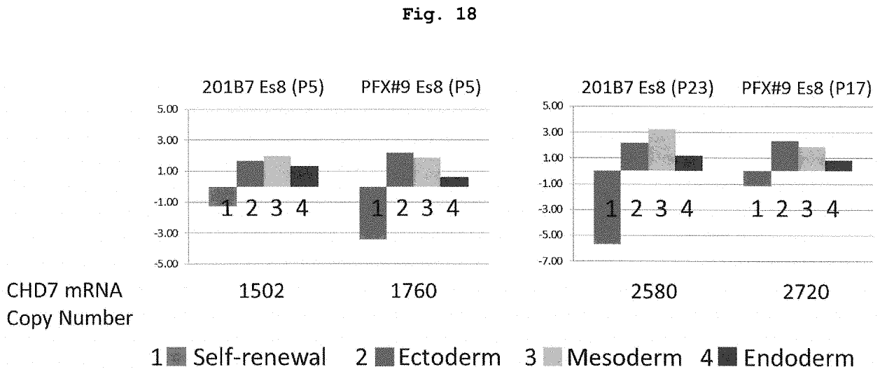

With regard to PSCs having differentiation potential, the threshold value of the copy number of CHD7 isoform 1 mRNA was further examined. Specifically, the differentiation potential of the cells made to have a lower copy number of CHD7 mRNA by culturing under conditions of overgrowth than normal (201B7 or PFX #9) was analyzed. The results of the copy number of CHD7 mRNA by digital PCR are shown in Table 8, and the results of differentiation potential are shown in FIG. 18.

TABLE 8

| | PFX#9 | 201B7 | Passage Method |
|---|---|---|---|
| Es8/VNT-N | 1760 (P5) | 1502 (P5) | Single Cell Suspension |
| | 2720 (P40) | 2580 (P23) | |

It was shown that 201B7 and PFX #9 have differentiation potential even when the copy number of CHD7 isoform 1 mRNA in 5 ng of total RNA was 1502 copies (201B7) or and 1760 copies (PFX #9) (FIG. 18). Based on the data of Table 7 and Table 8, and FIG. 17 and FIG. 18, a numerical criterion is proposed that when cultured at least feeder cell-free in single cells, in 5 ng of the total RNA, PSCs having a copy number of not more than 732 does not differentiate but PSCs having a copy number of not more than 1500 maintains differentiation potential. Therefore, the copy number of CHD7 isoform 1 of PSC may be a good numerical value marker for predicting the degree of differentiation property while maintaining PSCs in an undifferentiated state.

### 6. Expression Level of CHD7 Protein is Also a Prediction Marker of Differentiation Potential of PSCs:

To study not only the copy number of CHD7 mRNA but also the relationship between the expression level of CHD7 protein and the differentiation potential of PSCs, the measurement of the expression level of CHD7 protein was performed.

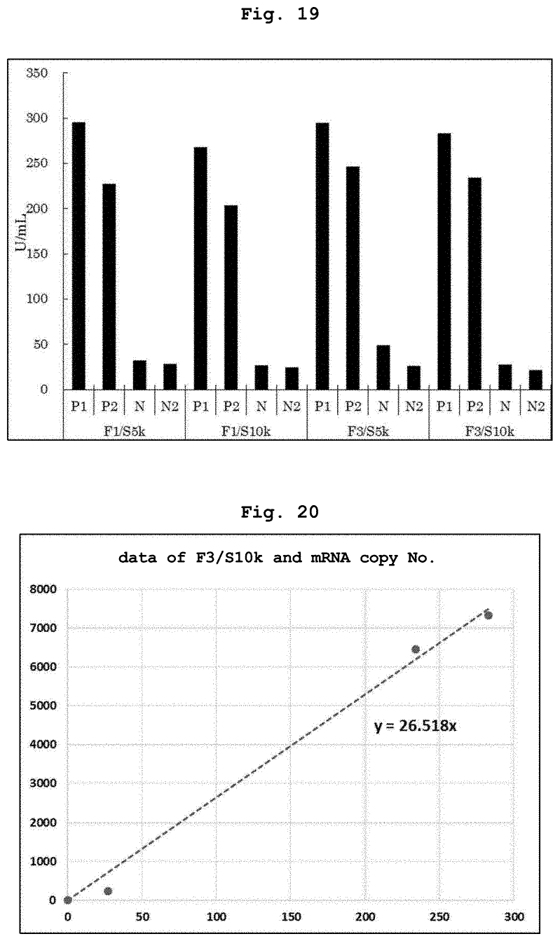

The culture conditions, the results of copy number of mRNA of CHD7 isoform 1 and differentiation potential of each cell are shown in Table 9, and the results of sandwich ELISA are shown in FIG. 19.

TABLE 9

| | | | Passage | CHD7 mRNA | |
|---|---|---|---|---|---|
| | Cell | Medium | Method | Copy No. | Differentiation |
| P1 | H9 | Essential 8 | Single Cell | 7340 (P17) | + |
| | | | Suspension | | |
| P2 | KhES | Essential 8 | Single Cell | 6460 (p10) | + |
| | | | Suspension | | |
| N | KhES | RFF2 | Single Cell | 232 (P11) | + |
| | | | Suspension | | |
| N2 | H9 | RFF2 | Single Cell | 316 (P17) | + |
| | | | Suspension | | |

The graph shows the values relative to the CHD7 protein concentration of a standard (same protein solution as P1 and concentrated to a concentration of 4.06 mg/mL using Amicon (registered trade mark) Ultra-4, PLTK Ultracel-PL membrane at 30 kDa (Millipor, UFC803024)) as 1000 Units/mL. In the case of F1/S5K, CHD7 protein concentrations of P1 and P2 were 9.2 times (N is 10.9% of P1) and 7.0 times (N is 14.2% of P2) as compared to the protein concentration of N. Similarly, in the case of F1/S10K, it was respectively 10.2 times (N is 9.8% of P1) and 7.7 times (N is 13.0% of P2), in the case of F3/S5K, it was respectively 6.0 times (N is 16.6% of P1) and 5.0 times (N is 19.8% of P2), and in the case of F3/S10K, it was respectively 10.3 times (N is 9.7% of P1) and 8.5 times (N is 11.7% of P2). As shown in FIG. 19, CHD7 protein concentration and the copy number of mRNA of CHD7 isoform 1 are low in differentiation-resistant PSCs: PSCs that show good differentiation potential in response to differentiation stimulus were confirmed to show high CHD7 protein concentration and high copy number of mRNA of CHD7 isoform 1, and a correlation was found between the expression level of CHD7 protein, and the copy number and differentiation potential of mRNA of CHD7 isoform 1 in PSCs. Not only the copy number of mRNA but also the expression level of CHD7 protein could be a good numerical marker for predicting the degree of differentiation property during PSCs are maintained in an undifferentiated state.

### Analysis Using CHD7 Protein Level and Expression Level of CHD7 Gene:

In consideration of the CHD7 protein level and gene expression level and using the above-mentioned results, the threshold value was determined.

TABLE 10
ELISA measured values

| | | concentration | sample/N | N/sample | 100-N/sample |
|---|---|---|---|---|---|
| label | sample | (U/mL) | (times) | (%) | (%) |
| F1/S5k | P1 | 295.4 | 9.2 | 10.9 | 89.1 |
| | P2 | 226.9 | 7.0 | 14.2 | 85.8 |
| | N | 32.2 | | | |
| | N2 | 28.2 | | | |
| F1/S10k | P1 | 267.4 | 10.2 | 9.8 | 90.2 |
| | P2 | 203.0 | 7.7 | 13.0 | 87.0 |
| | N | 26.3 | | | |
| | N2 | 24.2 | | | |
| F3/S5k | P1 | 294.3 | 6.0 | 16.6 | 83.4 |
| | P2 | 245.8 | 5.0 | 19.8 | 80.2 |
| | N | 48.8 | | | |
| | N2 | 25.5 | | | |
| F3/S10k | P1 | 283.0 | 10.3 | 9.7 | 90.3 |
| | P2 | 233.7 | 8.5 | 11.7 | 88.3 |
| | N | 27.4 | | | |
| | N2 | 21.2 | | | |

TABLE 11
Data of F3/S10k and mRNA copy No.

| | units/mL (X axis) | mRNA Copy No. (Y axis) |
|---|---|---|
| | 0 | 0 |
| N | 27 | 232 |
| P2 | 234 | 6460 |
| P1 | 283 | 7340 |

Asymptote was drawn from the mRNA copy number and the protein concentration (units/mL) obtained from the ELISA results (FIG. 20). When calculated using the asymptote, the concentration (x) was 56.6 units/mL when the copy number (y) in 5 ng of total RNA was 1500 copies. This was 2.1 times the concentration of N (N was 52% of the obtained concentration). Similarly, the concentration (x) was 102.2 units/mL when the copy number (y) in 5 ng of total RNA was 2710 copies. This was 3.8 times the concentration of N (N was 74% of the obtained concentration). In the DESCRIPTION, it is appreciated that a necessary appropriate threshold value can be obtained by using the asymptote also in copy number (y) which is other than those mentioned above and predicted to show differentiation potential in response to a differentiation stimulus.

### INDUSTRIAL APPLICABILITY

Whether PSC shows a differentiation potential in response to a differentiation stimulus can be predicted in an undifferentiated state before applying a differentiation stimulus by measuring the expression level of CHD7 in PSC. In addition, whether a medium used for maintenance culture of PSC is suitable for maintaining PSC in a state holding a property showing a differentiation potential in response to a differentiation stimulus can also be evaluated. Therefore, the present invention can be utilized for providing hPSC that shows no differentiation resistance during differentiation induction and has a reduced risk of tumorigenesis, and is extremely useful in a transplantation therapy using a hiPSC-derived differentiated cell. The present invention is also useful for searching for a medium and/or culture conditions suitable for maintenance culture of PSC so that differentiation resistance will not appear during differentiation induction.

This application is based on patent application No. 2017-120024 filed in Japan (filing date: Jun. 19, 2017) and patent application No. 2017-237420 filed in Japan (filing date: Dec. 12, 2017), the contents of which are incorporated in full herein.

1. A method for predicting a differentiation potential of a pluripotent stem cell comprising measuring an expression level of CHD7 of the human pluripotent stem cell.

2. The method according to claim 1, wherein a human pluripotent stem cell having the expression level of CHD7 of not less than 1500 copies in 5 ng of the total RNA is predicted to show a differentiation potential in response to a differentiation stimulus.

3. The method according to claim 2, wherein the expression level of CHD7 is not less than 2710 copies in 5 ng of the total RNA.

4. The method according to claim 1, wherein the expression level of CHD7 is an expression level of a human pluripotent stem cell cultured for not less than 5 passages with Essential 8 medium or Stem-Partner (registered trade mark) Human iPS/ES cells medium.

5. The method according to claim 1, wherein the human pluripotent stem cell is an embryonic stem cell or an induced pluripotent stem cell.

6. A method for evaluating a medium for a human pluripotent stem cell comprising measuring an expression level of CHD7 of the pluripotent stem cell.

7. The method according to claim 6, wherein the human pluripotent stem cell is a human pluripotent stem cell cultured for not less than 5 passages with the medium.

8. The method according to claim 6, wherein the medium is evaluated as being capable of maintaining the human pluripotent stem cell to show a differentiation potential in response to a differentiation stimulus, when the expression level of CHD7 is not less than 1500 copies in 5 ng of the total RNA.

9. The method according to claim 8, wherein the expression level of CHD7 is not less than 2710 copies in 5 ng of the total RNA.

10. The method according to claim 6, wherein the human pluripotent stem cell is an embryonic stem cell or an induced pluripotent stem cell.

11. A reagent or kit for predicting a differentiation potential of a human pluripotent stem cell and/or evaluating a medium for human pluripotent stem cell, comprising a substance capable of detecting an expression of CHD7.

12. (canceled)

13. The method according to claim 1, wherein the human pluripotent stem cell is predicted to show a differentiation potential in response to a differentiation stimulus, when the expression level of CHD7 is a protein level not less than two times a CHD7 protein level of a human pluripotent stem cell showing a differentiation resistance.

14. The method according to claim 13, wherein the expression level of CHD7 is an expression level of a human pluripotent stem cell cultured for not less than 5 passages with Essential 8 medium or Stem-Partner (registered trade mark) Human iPS/ES cells medium.

15. The method according to claim 13, wherein the human pluripotent stem cell showing the differentiation resistance is a human pluripotent stem cell cultured for not less than 5 passages with ReproFF2 medium.

16. The method according to claim 6, wherein the medium is evaluated as being capable of maintaining the human pluripotent stem cell to show a differentiation potential in response to a differentiation stimulus, when the expression level of CHD7 is a protein level not less than two times a CHD7 protein level of a human pluripotent stem cell showing a differentiation resistance.


SEQUENCE LISTING (30 sequences)

SEQ ID NO: 1 - DNA, 11589 bp, Homo sapiens
  CDS (493)..(9486)
gcggcggcggcggcggcggcggcggcagcggcggcggcggcggcggcgcgggggttgagtcgtggtggtgcggacgcgctcgtgctcgggaactatcggattaaacttgaatcgagtgaaattacacaaaggagcgccgcggaggaggcggcccggggacccggacaccctgaaactcaccagagacccgttcgcccccggccaactccgtgcccgtggattcagccccctggccgcagctgccgagccaactccggagcccgctctgcgttttgttttcccctcggcactaggcagcggaggagcccgaccgacccggacctatatccagactttgcctgacactgcagggtccaagagaattaaagaaatatggaatgacatgaagaagattagttaaggattataggctttgagggcaaacacctcagtgaagtgaagcacaggcaagctcctgagctgtggtttggaggagccgtgtgttggaagaagatggcagatccaggaatgatgagtctttttggcgaggatgggaatattttcagtgaaggtcttgaaggcctcggagaatgtggttacccggaaaatccagtaaatcctatgggtcagcaaatgccaatagaccaaggctttgcctctttacagccatcccttcatcatccttcaactaatcaaaatcaaacaaagctgacacattttgatcactataatcagtatgaacaacaaaagatgcatctgatggatcagccgaacagaatgatgagcaacacccctgggaacggactcgcgtctccgcactcgcagtatcacacccctcccgttcctcaggtgccccatggtggcagtggtggcggtcagatgggtgtctaccctggcatgcagaatgagaggcatgggcaatcctttgtggacagcagctccatgtggggccccagggctgttcaggtaccagaccagatacgagccccctaccagcagcagcagccacagccgcagccaccgcagccggctccgtcggggccccctgcacagggccaccctcagcacatgcagcagatgggcagctatatggcacgtggggatttttccatgcagcagcatggtcagccacagcagaggatgagccagttttcccaaggccaagagggcctcaatcagggaaatccttttattgccacctcaggacctggccacttgtcccacgtgccccagcagagtcccagcatggcaccttccttgcgtcactcggtgcagcagttccatcaccacccctctactgctctccatggagaatccgttgcccacagtcccagattctccccgaatcctccccaacaaggggctgttaggccgcaaacccttaactttagttctcggagccagacagtcccctctcctactataaacaactcagggcagtattctcgatatccttacagtaacctaaatcagggattagttaacaatacagggatgaatcaaaatttaggccttacaaataatactccaatgaatcagtccgtaccaagataccccaatgctgtaggattcccatcaaacagtggtcaaggactaatgcaccagcagcccatccaccccagtggctcacttaaccaaatgaacacacaaactatgcatccttcacagcctcagggaacttatgcctctccacctcccatgtcacccatgaaagcaatgagtaatccagcaggcactcctcctccacaagtcaggccgggaagtgctgggataccaatggaagttggcagttatccaaatatgccccatcctcagccatctcaccagccccctggtgccatgggaatcggacagaggaatatgggccccagaaacatgcagcagtctcgtccatttataggcatgtcctcggcaccaagggaattgactgggcacatgaggccaaatggttgtcctggtgttggccttggagacccacaagcaatccaggaacgactgatacctggccaacaacatcctggtcaacagccatcttttcagcagttgccaacctgtcctccactgcagcctcacccgggcttgcaccaccagtcttcacctccacaccctcatcaccagccttgggcacagctccacccatcaccccagaacaccccgcagaaagtgcctgtgcatcagcattccccgtcggagccctttctagagaaaccagtgccggatatgactcaggttagtggaccgaatgctcagctagtgaagagtgatgattacctgccatcaatagaacagcagccacaacaaaagaagaagaaaaagaaaaacaaccacattgtagcagaggatcccagtaaaggttttggtaaagatgacttccctggtggggtagataaccaagaactaaataggaactcactggatgggtcccaagaagaaaaaaagaaaaagaaaaggtcaaaggcaaaaaaagacccgaaggaaccgaaagaacccaaggagaaaaaagagcccaaggaacccaagaccccgaaagcccctaagattcccaaagagccaaaggaaaagaaagcaaaaactgccacgccaaaacccaaatccagcaaaaagtcaagtaataagaaacctgactcagaagcaagtgctttgaagaaaaaggtcaacaagggaaaaacagaaggttctgaaaattcagacttagacaaaacacccccaccatctcctcctcctgaagaagatgaggacccaggtgttcagaagagacggtccagcagacaggtgaagagaaagcgctacactgaagacctggagttcaagatttctgatgaggaggcagatgatgcagatgctgctgggagggattccccctccaacacctcccagtcagaacagcaggaatctgttgatgcagaaggcccagtggtagaaaaaattatgagcagtcgttcagtaaaaaagcagaaggaatctggagaggaggtagaaattgaggaattctatgtgaaatacaaaaacttctcttatcttcattgtcagtgggcatctatagaagatctggaaaaagataagagaattcagcaaaaaattaaacgatttaaggcaaagcagggccagaacaagttcctttcagagattgaggatgagctttttaatccagattatgtggaggttgaccggataatggactttgcacgtagcacagatgaccggggagagcctgtgactcactatctggtgaagtggtgttcacttccttatgaagacagcacgtgggagcggaggcaggacatagatcaagcaaagatcgaggagtttgagaaactaatgtccagggagccggaaacagagcgtgtggagcgacctcctgctgatgattggaagaaatcggagagttccagggagtataaaaacaataacaaactcagggaataccagttggagggagtaaactggctacttttcaattggtacaacatgcgaaactgcattttagcagatgaaatgggtttgggaaaaactatccagtccattacatttctctatgagatatatttgaaaggaatccatggcccttttttagtaattgccccattgtccacaatccccaactgggaaagggaattccgaacctggacagagttgaacgtggttgtgtatcatgggagtcaagctagtcgtcggaccattcagttgtatgaaatgtacttcaaagatccccagggtcgagtgataaaggggtcctataagtttcatgccatcatcactacatttgagatgattttgactgattgtcctgagctgcggaatattccatggcgctgtgtagtcattgatgaagcccacaggctgaagaacaggaactgcaagctgttggagggactcaagatgatggacttggaacacaaagtgctgctgacgggaaccccactccagaacactgtggaagaactcttcagcttgcttcatttcttggaaccaagtcgcttcccttcagaaaccacatttatgcaagaatttggtgatctaaaaacagaagagcaggtgcaaaaacttcaagctattctaaagccaatgatgttgagacgtctcaaagaggatgtagaaaagaacttggcccccaaagaagaaactattattgaagttgagctaacaaacattcagaagaaatattaccgagccatccttgagaagaatttcacatttctttccaaaggcggtggtcaagctaacgtacctaacctattaaacactatgatggaattgcggaagtgctgcaatcatccgtaccttatcaatggtgctgaagagaaaattttggaagagtttaaagaaacacacaatgcagagtctccagattttcagctccaggcaatgatccaggctgctggcaagctagtgctgattgacaagctgctgccaaaactgaaggctggtggccacagggtgcttatcttttcccagatggtgcgctgcttggacatactggaagactacctcattcaaagacggtacccatatgaaaggatcgacggccgagtaagaggcaacctccgccaggcagctatcgacagattctccaaacctgattctgataggtttgttttcctcctgtgtacaagggcaggaggtttaggcattaacctcactgctgctgatacctgcatcatctttgattcagactggaatccccaaaatgacctccaggctcaggctagatgtcatagaataggacagagcaaatctgtgaaaatctacaggctgattacaagaaattcctatgaaagggaaatgttcgacaaggctagtttgaaactgggcctggataaagctgtgctacagtctatgagtggaagagaaaatgctaccaatggggtacaacagctttccaagaaagaaatagaggatcttctacgaaaaggggcctatggtgcactcatggatgaggaggatgaagggtctaaattctgtgaagaagatattgatcagatcctcctacgtcgaacccacaccattaccattgagtcagaagggaaaggttccacatttgctaaggccagttttgttgcatctggaaataggacagatatttccttggatgatccaaatttctggcaaaagtgggctaagaaggctgaattggatattgatgccttaaatgggaggaacaacctggttattgatactccaagagtgagaaagcagaccaggctctacagtgcagtgaaggaagatgagctgatggagttctcagacttggaaagtgattctgaagaaaagccctgtgcaaagccacggcgtccccaggataagtcacagggctatgcaaggagtgaatgtttcagggtggagaagaatctgcttgtctatggttggggacggtggacagacattctttcccacggacgctataaacgccaactcactgagcaagatgtagaaaccatctgcagaaccatcctggtgtactgtcttaatcattacaaaggggatgagaatatcaaaagcttcatctgggatctgatcacacccacagcggatggccagactcgagccttggtcaaccattccggtttgtcagctcctgtgccaaggggaaggaagggaaagaaggtgaaagcccagagcacacagccggtggtgcaggatgccgactggctggccagctgcaacccagatgccctgttccaggaggacagctacaagaaacacctgaagcatcactgtaacaaggtcctgctgcgtgtccgcatgctgtactacctaagacaagaagtgataggagaccaggcggataagatcttagagggtgctgactcaagtgaagccgatgtgtggatccctgaacctttccatgctgaagttcctgcagattggtgggataaggaagcagacaaatccctcttaattggagtgttcaaacatggctatgagaagtacaactccatgcgagctgaccccgcgctgtgctttctggaacgagtcggtatgcctgatgccaaggccatagctgccgagcaaagaggaacagacatgctagcagatggtggtgacgggggagaatttgatagagaagatgaagacccagaatataaaccaaccagaacaccgttcaaagatgaaatagatgaatttgcaaattctccttcagaggataaggaagaatccatggaaatacatgccacaggcaagcacagtgagagtaatgctgagttaggccaactttactggcctaacacttcaaccctgactacacgtctgcgccggctcattactgcctatcagcgcagctataaaaggcaacagatgaggcaagaggccctaatgaagactgaccggcgcagacggcggcctcgagaggaagtgagagctctggaagcggaaagggaagctattatatctgagaagcggcaaaagtggacaagaagagaagaggctgatttttaccgtgtggtatccacctttggggttatttttgaccctgtgaaacagcaatttgactggaaccaatttagagcctttgccaggcttgacaaaaaatctgatgagagtttggagaaatacttcagttgttttgtggccatgtgtaggcgagtatgtcgaatgcccgtcaagccagatgatgaaccgcccgacctctcctccataattgagccgatcacagaggagcgagcctctcgaactctgtaccgcattgagctgctacggaagatccgcgagcaggttctccatcacccccagctgggagagaggcttaagctctgccagccaagcttggatctgccagagtggtgggagtgtggacggcatgaccgagacttgctggttggtgctgctaaacacggggtcagtcggacggattatcacatcctcaatgaccctgagttatccttcttggatgcacataaaaactttgctcaaaacagaggggcaggtaatacatcttccttgaacccactggcagttggatttgtccagactcctccagtcatctcatctgctcatattcaagatgagagggtactggaacaagccgaaggcaaagtggaggagcctgaaaacccagctgccaaggagaaatgtgagggcaaagaagaggaagaagaaaccgatggcagcgggaaggagagcaagcaggaatgtgaggcagaggccagctctgtgaaaaatgaactgaaaggtgttgaggtcggcgcagacactgggtccaaatctatttcagagaaaggttccgaagaggatgaagaggaaaagctggaggatgacgataagtcggaagagtcttcccagcccgaagcaggagctgtctctagagggaagaattttgatgaagaaagcaatgcttccatgagcactgctagagatgaaacccgagatggattctacatggaggacggagatccttcagtagctcagctccttcatgaaagaacatttgccttctcgttttggcctaaggatagagtaatgataaaccgcttagacaacatctgtgaagcagtgttgaaaggcaaatggccagtaaataggcgccagatgtttgatttccaaggcctcatcccaggttacacacccaccacagtggacagccccttgcagaagaggagctttgctgagctctccatggtcggccaagccagcattagtgggagtgaggacatcactacgtctcctcagttgtcaaaggaagatgccctcaacctctctgtccctcgccagcggaggaggaggaggagaaaaatcgaaattgaggccgaaagagctgccaagaggcgaaatctcatggagatggttgcccagcttcgagagtctcaggtggtctcagaaaatggacaagaaaaagttgtagatttatcaaaggcctcaagagaggcaacaagctctacctcaaatttttcatctctttcttcaaagtttatcttgcctaatgtctcaacaccagtgtctgatgcctttaagactcaaatggaactgctccaagcaggcctttcgcgcacacccacaaggcatctccttaatggctccctagtggatggagagcctcccatgaagaggaggcggggaaggaggaaaaatgtggagggacttgatctgcttttcatgagccacaaacggacgtcattgagtgcagaggatgctgaggtgaccaaagcttttgaagaagatatagagaccccaccaacaagaaacattccttctcccggacagctggacccagacacacggatccctgttatcaatcttgaagatgggactaggctggtgggggaagatgctcctaaaaataaggatttagttgaatggctgaagctgcaccctacttacactgttgatatgccaagttatgtaccaaagaatgcagatgtgctgttttcctcatttcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgcagttgctccagactggactgatatagttaagcagtctggttttgttcctgagtcgatgtttgaccgccttctcactgggcctgtagtgcggggagagggagcgagcagaagaggaagaaggcccaaaagtgagatcgccagagcagccgcggccgccgctgctgtggcctccacgtcagggatcaaccctttgctggtgaacagcctgtttgctggaatggacctgacgagccttcagaatctccagaatctccagtcgctccagctggcaggcctcatgggcttccctccaggactggcaacagctgccaccgccggaggcgatgcgaagaaccctgctgctgtgctgcccctgatgctgccaggaatggcgggcctgcccaacgtgtttggcttgggcgggctgttgaataaccctctgtcagctgctactggaaacaccactactgcttctagtcaaggagaaccggaagacagcacttcaaaaggagaggagaaaggaaatgagaatgaagacgagaacaaagactctgagaaaagcacagatgctgtttcggctgctgactctgcgaatggatctgttggtgctgctactgccccggctggattgccctcaaacccgctagccttcaaccctttcctcctgtccacaatggccccgggcctcttctacccatccatgtttctacctccaggactggggggattgacgctgcctgggttcccagcattggcaggacttcagaatgccgtgggctccagcgaagaaaaggctgctgacaaggctgagggaggaccctttaaagatggagagacccttgaaggcagcgatgccgaggagagcctggataagactgcagagtcctccctcttagaagacgaaatagcacagggtgaagagctagactcacttgatgggggggatgaaatagaaaacaatgaaaatgatgaataaccagtaccagttccagttcaagtgtttaaaacttttgacaagtggtagtcctactgtttacactcacagttaatgttcatacctagttttataagctgttctgtaacatagtgtagcaaaaaaaaaagttcaagtcatgttatacaggtgtgtcaaaaggtatcttggtcattaagtattgtgcagtgcattatttattatccctaggagagatgaaatttgagaggtgatcatgtctttttaaggaaacttacataatgctctgctttttttttttctcttggtaccattggtattataataaagagcaatttgtaactgagtggcactaatggaagaaagtgctgctcaaaggaagtatgaagttatatatttaattttttaattttaatttttaatttttttgctgtgaaggtcaagctgaaatttaccatacatatcatacttgctcatttgtttccctttttgactgtatgggggttcccacactcgtgcatacacacacatccatacactctgacaatctccacgctagtgtgaacgcctctgtcccgaggcgcagcaataataaggcagctgttgaatgtgaagggtccctttggaaaattaacctactgggagggttcttgccagacagaactacagttccattgtctcgtggtcttgtaatgcactggtaaaaacaaaataaatagatgaataaataaagagtgagagaagagagaatcaggtaccttttttaaattaaaggactttgttactttagccacaaagctaaaacagcattacctcagctctaaactagccttgaagtttacagacatgactttgtaaatgtattgtttttctttgttgtgatgtccttttatttttttctttgaaaactgctatcatgtaagataaaatgtaaattgctgccaactgtagtaatgatgcttttaataaaagtgacccatgatatgcagagatgtaattatagaatgtagtatatagggaaactggtgttcactctattttttttgtattcgcaatagatgcaaatacagcattctggcttttgtacagtttcttgatatatcctcttatactagattagcttttggtaagacactaaactgtctcgaagactagacaggaaggaaaaccttgaattactctcacataattccactccagatatctcaacagaaatgcatacaaaaagctcctattactccctcaaaagggcatctgagaccgagaatacttagaaatgtgtgcagcgtgtgataatgtggtacactgaagaacaaaagggcaaaagaaaaatgaggctttaacaggcacaatatctaggtcatttatccttggttaatgggtagaaaaacacaatgcggtagtgtcagcaagggacacaaaggcactctggtgtcctgcagaccagcgctcgatgccagaaaccagtgtgtggaaaaacccatgtggaattgaaacagacccacttaagcacgcacgcgcgcacgcacggtctcaggagctactgatttgtggacccctttttgacctttggtatttaaagtaaaatataatttgagatctactgttttcacctttttatgtcacctgaaccaacacaaagccatatttccatccagttaaaaagcaggggaagggatgtggacgagagtgtttcgtgtgtgttgccttcctccacacccttcccccaggacgtccgcacacagtccacctccagccagtgtgctgccagacaggtggtctaaattcctcccagactgtgagattcagttcgtttatgccccaagatgaaaatatgccccttaatgattctgggaaagaaacaacctgaaccctccaccttacagatcctgtgattgcttttatttatcatcgtcgttgtctgctgaaagtgaatgggccgtgcacactggaggaaagtgccttgaagagaatattttttgaaagggaattatttgaacacgggaaagtgaaactaggtctgcatgaagtataggaaatttaagtatttaagtaacaaagatgttagcagggagaatttgcttaataaaatgagtcattaaagggtgttttttttaaaaaaaaaaa

SEQ ID NO: 2 - protein, 2997 aa, Homo sapiens
MADPGMMSLFGEDGNIFSEGLEGLGECGYPENPVNPMGQQMPIDQGFASLQPSLHHPSTNQNQTKLTHFDHYNQYEQQKMHLMDQPNRMMSNTPGNGLASPHSQYHTPPVPQVPHGGSGGGQMGVYPGMQNERHGQSFVDSSSMWGPRAVQVPDQIRAPYQQQQPQPQPPQPAPSGPPAQGHPQHMQQMGSYMARGDFSMQQHGQPQQRMSQFSQGQEGLNQGNPFIATSGPGHLSHVPQQSPSMAPSLRHSVQQFHHHPSTALHGESVAHSPRFSPNPPQQGAVRPQTLNFSSRSQTVPSPTINNSGQYSRYPYSNLNQGLVNNTGMNQNLGLTNNTPMNQSVPRYPNAVGFPSNSGQGLMHQQPIHPSGSLNQMNTQTMHPSQPQGTYASPPPMSPMKAMSNPAGTPPPQVRPGSAGIPMEVGSYPNMPHPQPSHQPPGAMGIGQRNMGPRNMQQSRPFIGMSSAPRELTGHMRPNGCPGVGLGDPQAIQERLIPGQQHPGQQPSFQQLPTCPPLQPHPGLHHQSSPPHPHHQPWAQLHPSPQNTPQKVPVHQHSPSEPFLEKPVPDMTQVSGPNAQLVKSDDYLPSIEQQPQQKKKKKKNNHIVAEDPSKGFGKDDFPGGVDNQELNRNSLDGSQEEKKKKKRSKAKKDPKEPKEPKEKKEPKEPKTPKAPKIPKEPKEKKAKTATPKPKSSKKSSNKKPDSEASALKKKVNKGKTEGSENSDLDKTPPPSPPPEEDEDPGVQKRRSSRQVKRKRYTEDLEFKISDEEADDADAAGRDSPSNTSQSEQQESVDAEGPVVEKIMSSRSVKKQKESGEEVEIEEFYVKYKNFSYLHCQWASIEDLEKDKRIQQKIKRFKAKQGQNKFLSEIEDELFNPDYVEVDRIMDFARSTDDRGEPVTHYLVKWCSLPYEDSTWERRQDIDQAKIEEFEKLMSREPETERVERPPADDWKKSESSREYKNNNKLREYQLEGVNWLLFNWYNMRNCILADEMGLGKTIQSITFLYEIYLKGIHGPFLVIAPLSTIPNWEREFRTWTELNVVVYHGSQASRRTIQLYEMYFKDPQGRVIKGSYKFHAIITTFEMILTDCPELRNIPWRCVVIDEAHRLKNRNCKLLEGLKMMDLEHKVLLTGTPLQNTVEELFSLLHFLEPSRFPSETTFMQEFGDLKTEEQVQKLQAILKPMMLRRLKEDVEKNLAPKEETIIEVELTNIQKKYYRAILEKNFTFLSKGGGQANVPNLLNTMMELRKCCNHPYLINGAEEKILEEFKETHNAESPDFQLQAMIQAAGKLVLIDKLLPKLKAGGHRVLIFSQMVRCLDILEDYLIQRRYPYERIDGRVRGNLRQAAIDRFSKPDSDRFVFLLCTRAGGLGINLTAADTCIIFDSDWNPQNDLQAQARCHRIGQSKSVKIYRLITRNSYEREMFDKASLKLGLDKAVLQSMSGRENATNGVQQLSKKEIEDLLRKGAYGALMDEEDEGSKFCEEDIDQILLRRTHTITIESEGKGSTFAKASFVASGNRTDISLDDPNFWQKWAKKAELDIDALNGRNNLVIDTPRVRKQTRLYSAVKEDELMEFSDLESDSEEKPCAKPRRPQDKSQGYARSECFRVEKNLLVYGWGRWTDILSHGRYKRQLTEQDVETICRTILVYCLNHYKGDENIKSFIWDLITPTADGQTRALVNHSGLSAPVPRGRKGKKVKAQSTQPVVQDADWLASCNPDALFQEDSYKKHLKHHCNKVLLRVRMLYYLRQEVIGDQADKILEGADSSEADVWIPEPFHAEVPADWWDKEADKSLLIGVFKHGYEKYNSMRADPALCFLERVGMPDAKAIAAEQRGTDMLADGGDGGEFDREDEDPEYKPTRTPFKDEIDEFANSPSEDKEESMEIHATGKHSESNAELGQLYWPNTSTLTTRLRRLITAYQRSYKRQQMRQEALMKTDRRRRRPREEVRALEAEREAIISEKRQKWTRREEADFYRVVSTFGVIFDPVKQQFDWNQFRAFARLDKKSDESLEKYFSCFVAMCRRVCRMPVKPDDEPPDLSSIIEPITEERASRTLYRIELLRKIREQVLHHPQLGERLKLCQPSLDLPEWWECGRHDRDLLVGAAKHGVSRTDYHILNDPELSFLDAHKNFAQNRGAGNTSSLNPLAVGFVQTPPVISSAHIQDERVLEQAEGKVEEPENPAAKEKCEGKEEEEETDGSGKESKQECEAEASSVKNELKGVEVGADTGSKSISEKGSEEDEEEKLEDDDKSEESSQPEAGAVSRGKNFDEESNASMSTARDETRDGFYMEDGDPSVAQLLHERTFAFSFWPKDRVMINRLDNICEAVLKGKWPVNRRQMFDFQGLIPGYTPTTVDSPLQKRSFAELSMVGQASISGSEDITTSPQLSKEDALNLSVPRQRRRRRRKIEIEAERAAKRRNLMEMVAQLRESQVVSENGQEKVVDLSKASREATSSTSNFSSLSSKFILPNVSTPVSDAFKTQMELLQAGLSRTPTRHLLNGSLVDGEPPMKRRRGRRKNVEGLDLLFMSHKRTSLSAEDAEVTKAFEEDIETPPTRNIPSPGQLDPDTRIPVINLEDGTRLVGEDAPKNKDLVEWLKLHPTYTVDMPSYVPKNADVLFSSFQKPKQKRHRCRNPNKLDINTLTGEERVPVVNKRNGKKMGGAMAPPMKDLPRWLEENPEFAVAPDWTDIVKQSGFVPESMFDRLLTGPVVRGEGASRRGRRPKSEIARAAAAAAAVASTSGINPLLVNSLFAGMDLTSLQNLQNLQSLQLAGLMGFPPGLATAATAGGDAKNPAAVLPLMLPGMAGLPNVFGLGGLLNNPLSAATGNTTTASSQGEPEDSTSKGEEKGNENEDENKDSEKSTDAVSAADSANGSVGAATAPAGLPSNPLAFNPFLLSTMAPGLFYPSMFLPPGLGGLTLPGFPALAGLQNAVGSSEEKAADKAEGGPFKDGETLEGSDAEESLDKTAESSLLEDEIAQGEELDSLDGGDEIENNENDE

SEQ ID NO: 3 - DNA, 5124 bp, Homo sapiens
  CDS (175)..(3021)
gacctatatccagactttgcctgacactgcagggtccaagagaattaaagaaatatggaatgacatgaagaagattagttaaggattataggctttgagggcaaacacctcagtgaagtgaagcacaggcaagctcctgagctgtggtttggaggagccgtgtgttggaagaagatggcagatccaggaatgatgagtctttttggcgaggatgggaatattttcagtgaaggtcttgaaggcctcggagaatgtggttacccggaaaatccagtaaatcctatgggtcagcaaatgccaatagaccaaggctttgcctctttacagccatcccttcatcatccttcaactaatcaaaatcaaacaaagctgacacattttgatcactataatcagtatgaacaacaaaagatgcatctgatggatcagccgaacagaatgatgagcaacacccctgggaacggactcgcgtctccgcactcgcagtatcacacccctcccgttcctcaggtgccccatggtggcagtggtggcggtcagatgggtgtctaccctggcatgcagaatgagaggcatgggcaatcctttgtggacagcagctccatgtggggccccagggctgttcaggtaccagaccagatacgagccccctaccagcagcagcagccacagccgcagccaccgcagccggctccgtcggggccccctgcacagggccaccctcagcacatgcagcagatgggcagctatatggcacgtggggatttttccatgcagcagcatggtcagccacagcagaggatgagccagttttcccaaggccaagagggcctcaatcagggaaatccttttattgccacctcaggacctggccacttgtcccacgtgccccagcagagtcccagcatggcaccttccttgcgtcactcggtgcagcagttccatcaccacccctctactgctctccatggagaatccgttgcccacagtcccagattctccccgaatcctccccaacaaggggctgttaggccgcaaacccttaactttagttctcggagccagacagtcccctctcctactataaacaactcagggcagtattctcgatatccttacagtaacctaaatcagggattagttaacaatacagggatgaatcaaaatttaggccttacaaataatactccaatgaatcagtccgtaccaagataccccaatgctgtaggattcccatcaaacagtggtcaaggactaatgcaccagcagcccatccaccccagtggctcacttaaccaaatgaacacacaaactatgcatccttcacagcctcagggaacttatgcctctccacctcccatgtcacccatgaaagcaatgagtaatccagcaggcactcctcctccacaagtcaggccgggaagtgctgggataccaatggaagttggcagttatccaaatatgccccatcctcagccatctcaccagccccctggtgccatgggaatcggacagaggaatatgggccccagaaacatgcagcagtctcgtccatttataggcatgtcctcggcaccaagggaattgactgggcacatgaggccaaatggttgtcctggtgttggccttggagacccacaagcaatccaggaacgactgatacctggccaacaacatcctggtcaacagccatcttttcagcagttgccaacctgtcctccactgcagcctcacccgggcttgcaccaccagtcttcacctccacaccctcatcaccagccttgggcacagctccacccatcaccccagaacaccccgcagaaagtgcctgtgcatcagcattccccgtcggagccctttctagagaaaccagtgccggatatgactcagaaaccgaaacagaaacgacatagatgtcgaaaccctaataaattggatataaacactttgacaggagaagaaagggtgcctgttgtcaataaacgaaatgggaagaagatgggtggagctatggcgcctccaatgaaggatctacccaggtggctggaagaaaatcctgaatttgcagttgctccagactggactgatatagttaagcagtctggttttgttcctgagtcgatgtttgaccgccttctcactgggcctgtagtgcggggagagggagcgagcagaagaggaagaaggcccaaaagtgagatcgccagagcagccgcggccgccgctgctgtggcctccacgtcagggatcaaccctttgctggtgaacagcctgtttgctggaatggacctgacgagccttcagaatctccagaatctccagtcgctccagctggcaggcctcatgggcttccctccaggactggcaacagctgccaccgccggaggcgatgcgaagaaccctgctgctgtgctgcccctgatgctgccaggaatggcgggcctgcccaacgtgtttggcttgggcgggctgttgaataaccctctgtcagctgctactggaaacaccactactgcttctagtcaaggagaaccggaagacagcacttcaaaaggagaggagaaaggaaatgagaatgaagacgagaacaaagactctgagaaaagcacagatgctgtttcggctgctgactctgcgaatggatctgttggtgctgctactgccccggctggattgccctcaaacccgctagccttcaaccctttcctcctgtccacaatggccccgggcctcttctacccatccatgtttctacctccaggactggggggattgacgctgcctgggttcccagcattggcaggacttcagaatgccgtgggctccagcgaagaaaaggctgctgacaaggctgagggaggaccctttaaagatggagagacccttgaaggcagcgatgccgaggagagcctggataagactgcagagtcctccctcttagaagacgaaatagcacagggtgaagagctagactcacttgatgggggggatgaaatagaaaacaatgaaaatgatgaataaccagtaccagttccagttcaagtgtttaaaacttttgacaagtggtagtcctactgtttacactcacagttaatgttcatacctagttttataagctgttctgtaacatagtgtagcaaaaaaaaaagttcaagtcatgttatacaggtgtgtcaaaaggtatcttggtcattaagtattgtgcagtgcattatttattatccctaggagagatgaaatttgagaggtgatcatgtctttttaaggaaacttacataatgctctgctttttttttttctcttggtaccattggtattataataaagagcaatttgtaactgagtggcactaatggaagaaagtgctgctcaaaggaagtatgaagttatatatttaattttttaattttaatttttaatttttttgctgtgaaggtcaagctgaaatttaccatacatatcatacttgctcatttgtttccctttttgactgtatgggggttcccacactcgtgcatacacacacatccatacactctgacaatctccacgctagtgtgaacgcctctgtcccgaggcgcagcaataataaggcagctgttgaatgtgaagggtccctttggaaaattaacctactgggagggttcttgccagacagaactacagttccattgtctcgtggtcttgtaatgcactggtaaaaacaaaataaatagatgaataaataaagagtgagagaagagagaatcaggtaccttttttaaattaaaggactttgttactttagccacaaagctaaaacagcattacctcagctctaaactagccttgaagtttacagacatgactttgtaaatgtattgtttttctttgttgtgatgtccttttatttttttctttgaaaactgctatcatgtaagataaaatgtaaattgctgccaactgtagtaatgatgcttttaataaaagtgacccatgatatgcagagatgtaattatagaatgtagtatatagggaaactggtgttcactctattttttttgtattcgcaatagatgcaaatacagcattctggcttttgtacagtttcttgatatatcctcttatactagattagcttttggtaagacactaaactgtctcgaagactagacaggaaggaaaaccttgaattactctcacataattccactccagatatctcaacagaaatgcatacaaaaagctcctattactccctcaaaagggcatctgagaccgagaatacttagaaatgtgtgcagcgtgtgataatgtggtacactgaagaacaaaagggcaaaagaaaaatgaggctttaacaggcacaatatctaggtcatttatccttggttaatgggtagaaaaacacaatgcggtagtgtcagcaagggacacaaaggcactctggtgtcctgcagaccagcgctcgatgccagaaaccagtgtgtggaaaaacccatgtggaattgaaacagacccacttaagcacgcacgcgcgcacgcacggtctcaggagctactgatttgtggacccctttttgacctttggtatttaaagtaaaatataatttgagatctactgttttcacctttttatgtcacctgaaccaacacaaagccatatttccatccagttaaaaagcaggggaagggatgtggacgagagtgtttcgtgtgtgttgccttcctccacacccttcccccaggacgtccgcacacagtccacctccagccagtgtgctgccagacaggtggtctaaattcctcccagactgtgagattcagttcgtttatgccccaagatgaaaatatgccccttaatgattctgggaaagaaacaacctgaaccctccaccttacagatcctgtgattgcttttatttatcatcgtcgttgtctgctgaaagtgaatgggccgtgcacactggaggaaagtgccttgaagagaatattttttgaaagggaattatttgaacacgggaaagtgaaactaggtctgcatgaagtataggaaatttaagtatttaagtaacaaagatgttagcagggagaatttgcttaataaaatgagtcattaaagggtgttttttttaaaaaaaaaaa

SEQ ID NO: 4 - protein, 948 aa, Homo sapiens
MADPGMMSLFGEDGNIFSEGLEGLGECGYPENPVNPMGQQMPIDQGFASLQPSLHHPSTNQNQTKLTHFDHYNQYEQQKMHLMDQPNRMMSNTPGNGLASPHSQYHTPPVPQVPHGGSGGGQMGVYPGMQNERHGQSFVDSSSMWGPRAVQVPDQIRAPYQQQQPQPQPPQPAPSGPPAQGHPQHMQQMGSYMARGDFSMQQHGQPQQRMSQFSQGQEGLNQGNPFIATSGPGHLSHVPQQSPSMAPSLRHSVQQFHHHPSTALHGESVAHSPRFSPNPPQQGAVRPQTLNFSSRSQTVPSPTINNSGQYSRYPYSNLNQGLVNNTGMNQNLGLTNNTPMNQSVPRYPNAVGFPSNSGQGLMHQQPIHPSGSLNQMNTQTMHPSQPQGTYASPPPMSPMKAMSNPAGTPPPQVRPGSAGIPMEVGSYPNMPHPQPSHQPPGAMGIGQRNMGPRNMQQSRPFIGMSSAPRELTGHMRPNGCPGVGLGDPQAIQERLIPGQQHPGQQPSFQQLPTCPPLQPHPGLHHQSSPPHPHHQPWAQLHPSPQNTPQKVPVHQHSPSEPFLEKPVPDMTQKPKQKRHRCRNPNKLDINTLTGEERVPVVNKRNGKKMGGAMAPPMKDLPRWLEENPEFAVAPDWTDIVKQSGFVPESMFDRLLTGPVVRGEGASRRGRRPKSEIARAAAAAAAVASTSGINPLLVNSLFAGMDLTSLQNLQNLQSLQLAGLMGFPPGLATAATAGGDAKNPAAVLPLMLPGMAGLPNVFGLGGLLNNPLSAATGNTTTASSQGEPEDSTSKGEEKGNENEDENKDSEKSTDAVSAADSANGSVGAATAPAGLPSNPLAFNPFLLSTMAPGLFYPSMFLPPGLGGLTLPGFPALAGLQNAVGSSEEKAADKAEGGPFKDGETLEGSDAEESLDKTAESSLLEDEIAQGEELDSLDGGDEIENNENDE

SEQ ID NO: 5 - DNA, 5465 bp, Homo sapiens
  CDS (514)..(5379)
aggcgctggcgtgctggggccgcggcggcggcggcggcggcggcggcagcggcggcggcggcggcggcgcgggggttgagtcgtggtggtgcggacgcgctcgtgctcgggaactatcggattaaacttgaatcgagtgaaattacacaaaggagcgccgcggaggaggcggcccggggacccggacaccctgaaactcaccagagacccgttcgcccccggccaactccgtgcccgtggattcagccccctggccgcagctgccgagccaactccggagcccgctctgcgttttgttttcccctcggcactaggcagcggaggagcccgaccgacccggacctatatccagactttgcctgacactgcagggtccaagagaattaaagaaatatggaatgacatgaagaagattagttaaggattataggctttgagggcaaacacctcagtgaagtgaagcacaggcaagctcctgagctgtggtttggaggagccgtgtgttggaagaagatggcagatccaggaatgatgagtctttttggcgaggatgggaatattttcagtgaaggtcttgaaggcctcggagaatgtggttacccggaaaatccagtaaatcctatgggtcagcaaatgccaatagaccaaggctttgcctctttacagccatcccttcatcatccttcaactaatcaaaatcaaacaaagctgacacattttgatcactataatcagtatgaacaacaaaagatgcatctgatggatcagccgaacagaatgatgagcaacacccctgggaacggactcgcgtctccgcactcgcagtatcacacccctcccgttcctcaggtgccccatggtggcagtggtggcggtcagatgggtgtctaccctggcatgcagaatgagaggcatgggcaatcctttgtggacagcagctccatgtggggccccagggctgttcaggtaccagaccagatacgagccccctaccagcagcagcagccacagccgcagccaccgcagccggctccgtcggggccccctgcacagggccaccctcagcacatgcagcagatgggcagctatatggcacgtggggatttttccatgcagcagcatggtcagccacagcagaggatgagccagttttcccaaggccaagagggcctcaatcagggaaatccttttattgccacctcaggacctggccacttgtcccacgtgccccagcagagtcccagcatggcaccttccttgcgtcactcggtgcagcagttccatcaccacccctctactgctctccatggagaatccgttgcccacagtcccagattctccccgaatcctccccaacaaggggctgttaggccgcaaacccttaactttagttctcggagccagacagtcccctctcctactataaacaactcagggcagtattctcgatatccttacagtaacctaaatcagggattagttaacaatacagggatgaatcaaaatttaggccttacaaataatactccaatgaatcagtccgtaccaagataccccaatgctgtaggattcccatcaaacagtggtcaaggactaatgcaccagcagcccatccaccccagtggctcacttaaccaaatgaacacacaaactatgcatccttcacagcctcagggaacttatgcctctccacctcccatgtcacccatgaaagcaatgagtaatccagcaggcactcctcctccacaagtcaggccgggaagtgctgggataccaatggaagttggcagttatccaaatatgccccatcctcagccatctcaccagccccctggtgccatgggaatcggacagaggaatatgggccccagaaacatgcagcagtctcgtccatttataggcatgtcctcggcaccaagggaattgactgggcacatgaggccaaatggttgtcctggtgttggccttggagacccacaagcaatccaggaacgactgatacctggccaacaacatcctggtcaacagccatcttttcagcagttgccaacctgtcctccactgcagcctcacccgggcttgcaccaccagtcttcacctccacaccctcatcaccagccttgggcacagctccacccatcaccccagaacaccccgcagaaagtgcctgtgcatcagcattccccgtcggagccctttctagagaaaccagtgccggatatgactcaggttagtggaccgaatgctcagctagtgaagagtgatgattacctgccatcaatagaacagcagccacaacaaaagaagaagaaaaagaaaaacaaccacattgtagcagaggatcccagtaaaggttttggtaaagatgacttccctggtggggtagataaccaagaactaaataggaactcactggatgggtcccaagaagaaaaaaagaaaaagaaaaggtcaaaggcaaaaaaagacccgaaggaaccgaaagaacccaaggagaaaaaagagcccaaggaacccaagaccccgaaagcccctaagattcccaaagagccaaaggaaaagaaagcaaaaactgccacgccaaaacccaaatccagcaaaaagtcaagtaataagaaacctgactcagaagcaagtgctttgaagaaaaaggtcaacaagggaaaaacagaaggttctgaaaattcagacttagacaaaacacccccaccatctcctcctcctgaagaagatgaggacccaggtgttcagaagagacggtccagcagacaggtgaagagaaagcgctacactgaagacctggagttcaagatttctgatgaggaggcagatgatgcagatgctgctgggagggattccccctccaacacctcccagtcagaacagcaggaatctgttgatgcagaaggcccagtggtagaaaaaattatgagcagtcgttcagtaaaaaagcagaaggaatctggagaggaggtagaaattgaggaattctatgtgaaatacaaaaacttctcttatcttcattgtcagtgggcatctatagaagatctggaaaaagataagagaattcagcaaaaaattaaacgatttaaggcaaagcagggccagaacaagttcctttcagagattgaggatgagctttttaatccagattatgtggaggttgaccggataatggactttgcacgtagcacagatgaccggggagagcctgtgactcactatctggtgaagtggtgttcacttccttatgaagacagcacgtgggagcggaggcaggacatagatcaagcaaagatcgaggagtttgagaaactaatgtccagggagccggaaacagagcgtgtggagcgacctcctgctgatgattggaagaaatcggagagttccagggagtataaaaacaataacaaactcagggaataccagttggagggagtaaactggctacttttcaattggtacaacatgcgaaactgcattttagcagatgaaatgggtttgggaaaaactatccagtccattacatttctctatgagatatatttgaaaggaatccatggcccttttttagtaattgccccattgtccacaatccccaactgggaaagggaattccgaacctggacagagttgaacgtggttgtgtatcatgggagtcaagctagtcgtcggaccattcagttgtatgaaatgtacttcaaagatccccagggtcgagtgataaaggggtcctataagtttcatgccatcatcactacatttgagatgattttgactgattgtcctgagctgcggaatattccatggcgctgtgtagtcattgatgaagcccacaggctgaagaacaggaactgcaagctgttggagggactcaagatgatggacttggaacacaaagtgctgctgacgggaaccccactccagaacactgtggaagaactcttcagcttgcttcatttcttggaaccaagtcgcttcccttcagaaaccacatttatgcaagaatttggtgatctaaaaacagaagagcaggtgcaaaaacttcaagctattctaaagccaatgatgttgagacgtctcaaagaggatgtagaaaagaacttggcccccaaagaagaaactattattgaagttgagctaacaaacattcagaagaaatattaccgagccatccttgagaagaatttcacatttctttccaaaggcggtggtcaagctaacgtacctaacctattaaacactatgatggaattgcggaagtgctgcaatcatccgtaccttatcaatggtgctgaagagaaaattttggaagagtttaaagaaacacacaatgcagagtctccagattttcagctccaggcaatgatccaggctgctggcaagctagtgctgattgacaagctgctgccaaaactgaaggctggtggccacagggtgcttatcttttcccagatggtgcgctgcttggacatactggaagactacctcattcaaagacggtacccatatgaaaggatcgacggccgagtaagaggcaacctccgccaggcagctatcgacagattctccaaacctgattctgataggtttgttttcctcctgtgtacaagggcaggaggtttaggcattaacctcactgctgctgatacctgcatcatctttgattcagactggaatccccaaaatgacctccaggctcaggctagatgtcatagaataggacagagcaaatctgtgaaaatctacaggctgattacaagaaattcctatgaaagggaaatgttcgacaaggctagtttgaaactgggcctggataaagctgtgctacagtctatgagtggaagagaaaatgctaccaatggggtacaacagctttccaagaaagaaatagaggatcttctacgaaaaggggcctatggtgcactcatggatgaggaggatgaagggtctaaattctgtgaagaagatattgatcagatcctcctacgtcgaacccacaccattaccattgagtcagaagggaaaggttccacatttgctaaggccagttttgttgcatctggaaataggacagatatttccttggatgatccaaatttctggcaaaagtgggctaagaaggctgaattggatattgatgccttaaatgggaggaacaacctggttattgatactccaagagtgagaaagcagaccaggctctacagtgcagtgaaggaagatgagctgatggagttctcagacttggaaagtgattctgaagaaaagccctgtgcaaagccacggcgtccccaggataagtcacagggctatgcaaggagtgaatgtttcagggtggagaagaatctgcttgtctatggtgtgaattgttgctagttttgacccaggatttcccaacgctggtacctgacttaaagtaaagccataaatcaaaactcacaggcacctctgcatgctggata

SEQ ID NO: 6 - protein, 1621 aa, Homo sapiens
MADPGMMSLFGEDGNIFSEGLEGLGECGYPENPVNPMGQQMPIDQGFASLQPSLHHPSTNQNQTKLTHFDHYNQYEQQKMHLMDQPNRMMSNTPGNGLASPHSQYHTPPVPQVPHGGSGGGQMGVYPGMQNERHGQSFVDSSSMWGPRAVQVPDQIRAPYQQQQPQPQPPQPAPSGPPAQGHPQHMQQMGSYMARGDFSMQQHGQPQQRMSQFSQGQEGLNQGNPFIATSGPGHLSHVPQQSPSMAPSLRHSVQQFHHHPSTALHGESVAHSPRFSPNPPQQGAVRPQTLNFSSRSQTVPSPTINNSGQYSRYPYSNLNQGLVNNTGMNQNLGLTNNTPMNQSVPRYPNAVGFPSNSGQGLMHQQPIHPSGSLNQMNTQTMHPSQPQGTYASPPPMSPMKAMSNPAGTPPPQVRPGSAGIPMEVGSYPNMPHPQPSHQPPGAMGIGQRNMGPRNMQQSRPFIGMSSAPRELTGHMRPNGCPGVGLGDPQAIQERLIPGQQHPGQQPSFQQLPTCPPLQPHPGLHHQSSPPHPHHQPWAQLHPSPQNTPQKVPVHQHSPSEPFLEKPVPDMTQVSGPNAQLVKSDDYLPSIEQQPQQKKKKKKNNHIVAEDPSKGFGKDDFPGGVDNQELNRNSLDGSQEEKKKKKRSKAKKDPKEPKEPKEKKEPKEPKTPKAPKIPKEPKEKKAKTATPKPKSSKKSSNKKPDSEASALKKKVNKGKTEGSENSDLDKTPPPSPPPEEDEDPGVQKRRSSRQVKRKRYTEDLEFKISDEEADDADAAGRDSPSNTSQSEQQESVDAEGPVVEKIMSSRSVKKQKESGEEVEIEEFYVKYKNFSYLHCQWASIEDLEKDKRIQQKIKRFKAKQGQNKFLSEIEDELFNPDYVEVDRIMDFARSTDDRGEPVTHYLVKWCSLPYEDSTWERRQDIDQAKIEEFEKLMSREPETERVERPPADDWKKSESSREYKNNNKLREYQLEGVNWLLFNWYNMRNCILADEMGLGKTIQSITFLYEIYLKGIHGPFLVIAPLSTIPNWEREFRTWTELNVVVYHGSQASRRTIQLYEMYFKDPQGRVIKGSYKFHAIITTFEMILTDCPELRNIPWRCVVIDEAHRLKNRNCKLLEGLKMMDLEHKVLLTGTPLQNTVEELFSLLHFLEPSRFPSETTFMQEFGDLKTEEQVQKLQAILKPMMLRRLKEDVEKNLAPKEETIIEVELTNIQKKYYRAILEKNFTFLSKGGGQANVPNLLNTMMELRKCCNHPYLINGAEEKILEEFKETHNAESPDFQLQAMIQAAGKLVLIDKLLPKLKAGGHRVLIFSQMVRCLDILEDYLIQRRYPYERIDGRVRGNLRQAAIDRFSKPDSDRFVFLLCTRAGGLGINLTAADTCIIFDSDWNPQNDLQAQARCHRIGQSKSVKIYRLITRNSYEREMFDKASLKLGLDKAVLQSMSGRENATNGVQQLSKKEIEDLLRKGAYGALMDEEDEGSKFCEEDIDQILLRRTHTITIESEGKGSTFAKASFVASGNRTDISLDDPNFWQKWAKKAELDIDALNGRNNLVIDTPRVRKQTRLYSAVKEDELMEFSDLESDSEEKPCAKPRRPQDKSQGYARSECFRVEKNLLVYGVNCC

SEQ ID NO: 7 - DNA, 20 bp, Artificial Sequence
  primer
gaaacccacactgcagcaga

SEQ ID NO: 8 - DNA, 18 bp, Artificial Sequence
  primer
tcgcttgcccttctggcg

SEQ ID NO: 9 - DNA, 26 bp, Artificial Sequence
  primer
gggaaatgggaggggtgcaaaagagg

SEQ ID NO: 10 - DNA, 26 bp, Artificial Sequence
  primer
ttgcgtgagtgtggatgggattggtg

SEQ ID NO: 11 - DNA, 23 bp, Artificial Sequence
  primer
ctcagctacaaacaggtgaagac

SEQ ID NO: 12 - DNA, 23 bp, Artificial Sequence
  primer
tccctggtggtaggaagagtaaa

SEQ ID NO: 13 - DNA, 23 bp, Artificial Sequence
  primer
ttgaatgtacagagtgcggaaga

SEQ ID NO: 14 - DNA, 22 bp, Artificial Sequence
  primer
aacagccatcacagacgaatcc

SEQ ID NO: 15 - DNA, 20 bp, Artificial Sequence
  primer
ggttcccacactcgtgcata

SEQ ID NO: 16 - DNA, 16 bp, Artificial Sequence
  primer
tgcgcctcgggacaga

SEQ ID NO: 17 - DNA, 23 bp, Artificial Sequence
  primer
cccatgaaagcaatgagtaatcc

SEQ ID NO: 18 - DNA, 22 bp, Artificial Sequence
  primer
tccattggtatcccagcacttc

SEQ ID NO: 19 - DNA, 23 bp, Artificial Sequence
  primer
tgatggacttggaacacaaagtg

SEQ ID NO: 20 - DNA, 20 bp, Artificial Sequence
  primer
tgaagggaagcgacttggtt

SEQ ID NO: 21 - DNA, 27 bp, Artificial Sequence
  primer
caaacatggctatgagaagtacaactc

SEQ ID NO: 22 - DNA, 20 bp, Artificial Sequence
  primer
ccgactcgttccagaaagca

SEQ ID NO: 23 - DNA, 20 bp, Artificial Sequence
  primer
ccactcctccacctttgacg

SEQ ID NO: 24 - DNA, 20 bp, Artificial Sequence
  primer
atgaggtccaccaccctgtt

SEQ ID NO: 25 - DNA, 30 bp, Artificial Sequence
  probe
tatgactcagaaaccgaaacagaaacgaca

SEQ ID NO: 26 - DNA, 22 bp, Artificial Sequence
  primer
gccctttctagagaaaccagtg

SEQ ID NO: 27 - DNA, 20 bp, Artificial Sequence
  primer
aggcaccctttcttctcctg

SEQ ID NO: 28 - DNA, 28 bp, Artificial Sequence
  probe
cacggacgctataaacgccaactcactg

SEQ ID NO: 29 - DNA, 23 bp, Artificial Sequence
  primer
gaatctgcttgtctatggttggg

SEQ ID NO: 30 - DNA, 20 bp, Artificial Sequence
  primer
aggatggttctgcagatggt